SEQUENCE LISTING (29 sequences)

SEQ ID NO: 1 - protein, 565 aa, Mus musculus
MDPEPTEHSTDGVSVPRQPPSAQTGLDVQVVSAAGDSGTMSQDTEVDMKDVELNELEPEKQPMNAADGAAAGEKNGLVKIKVAEDETEAGVKFTGLSKEELLKVAGSPGWVRTRWALLLLFWLGWLGMLAGAVVIIVRAPRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVA

SEQ ID NO: 2 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 3 - DNA, 3417 bp, synthetic construct
  source 1..3417: mol_type = other DNA
taagtacactgtagctgtcctcagacactccagaagagggcgtcagatctcgttacagatggttgtgagccaccatgtggttgctgggatttgaactcctgaccttcggaagagcagtcgggtgctcttacccactgagccatctcaccagcccctactttgaacttttcatgcctccacttcctggggttttaggcctgtgcctcctctgtgcctggtttatgtggtgctaaggatagaactcagggctttcaaatgctaagcaagcactggctgagctccacccccagtccttcatccttcactgctttggtgttgcagattgaaccctgggccttgcacatggtaggaaagcaccggtcttctgaactgcatcgtcagtcctttaagtttatatagtcaactgtgttagttattgtgttctatccctggtaagggaaagcaatctacgtagagcattgagagatgctgtcacttgagtcagagcttgaaggaggaagagtaacttagcaggaagatccaaacagcaggacagcatgcacaggaggcattaagaatggaaacaccctgtgtgtcgtgactctggcaggtgctagaaagctgctgtggctgaacagtagtgattaggaagggtgctgctgaaaagactgatgggagctcacgtctcctctctaagatgcttaggaatagatttctgccacttggaggtgggggcggggagatactgtgttaccttaaattttacttactttgtggagggcagtgcgcatgtgtggagggcagtgcgcatgtgtggagggcagaagacagctcctgggaatcagttctcctttcactgatcttcactgatcactgaggagtgagctcaggtttgctggctaggcaccttcaccagaggaacatcttttttctctttttggtctttttttttttaaagatttatttattcattatatgtaagtacactgtagctgtcttcagacgctctcgttaatggatggttgtgagccaccatgtggttgctgggatttgaactccggaccttcagaagagcagtagggtgctcttacccactgagccatctcaccagccctttggtttggttttttgagacagggtttctctgtgtagccatggctgtcctggaactcactctgtagaccaggctggcctcgaactcagagatccacctgcctctgcctcccgagtgctgggatcaaaggtgtgcgccaccacgcccggctccagatgaacatctggttggtttgatccatcttgttttcagtttggttgattgaaacaccatgtttctaatgagcatttcatcaaaccctttcaaggagtgcttctgcttcacagtccacaaaccttgagctttcacgctgtgtataaactctgcattggacttttcttatctgtcatgcccagggcaaggtaggactcttatcaactatgaacactagcatgacccttagcctcttccctcttcctaggagctggtgtatagacttttctctagtgaggccggggaggactttgggaattcaaaaggacagtggcaggtgatgagacttttcatatcccttcctctctactgttctggccttggcttctgctttcttccaaatacatagcaatcttggctgccggcctacgtggggagagggagaaaggacctgttgtttccttccgattctctctgaaaataacctaattggaacacaaagttcctgaaattgttggcacctcctagagttgatgttcacttttcacttgtccctgctgctaacccagtctgccccatttacccttcctcttgccaaggacctctgccgggggaatttgagaaactttcaaagtgtgagggaactaagctggttccagggcctagcatagtcaactgatgtagcccggcacactagagggtttgagagttattgcaaggtggttaagagttagaacaggggctggagagatggctcagcagttaagagcactcactgctctttcaaaggtcccgagttcaattcccaggaaccacatggtggctcacaaccatctgtaacgagttctgacgccctcttctggtacatctgaagacagctacagtgtagtcacatataataagtgagtaaatagttcttctggtgcatctgaagacagctacagtgtagtcacatataataagtgagtaaatagttaaaaaatagtaaatagttaaaaaaaaaagaaaaaaagagttagaacagcttgtatattaaaagggtaggtgcctcaataccttcggaatgtttattacagccccattaatctcttctggatgcctgattcccagtaatccaacattcttgctagtaactagggtaagataataagggtagtgaccacttcaattctgcagttgaattttaaaatcaagaagaacggattagagttgatcctatgcagttgatgaagcaaaggattggagtgggtgcccagtaaatagttgctgcctgaggaattaaattcaaggagtcttgaaaggtgagtacgtgtaagccccgaggagaatccaggaactcagctaagtccggagtatgagaagatgtacgtaaagcagaagtgagatgtagtgccaaggtctgtgtgaagttggggagatcagcctgagccgtgcttggggcgaagggatcacgatgtggatggagactggtgtcatcttgtggagagaaaagaggcctcgcagggtaaggggccggaaggtcggaggaggggctttgaaacctggtcaagacaaattcacgtttgttattctaagggtttctgaccacaagctccccttcccagactcttgcaggaataagcgttagggatagacgggagtattcagcgaggctggaggtgcgaaaagacttctctgcatctctttaagaggcggaccggggcggagtcctgggtcacttccccgccgccacacccgcccagcggcagaagcagttaggaagctctgctagcctcacggccacgggacgcctctctgaacggggatccaggcaggattagagctgcctcactgactacaggccgtgtcgtgtcaccgtttctgcaggcaccatgagccaggacaccgaagtggacatgaaagatgtggagctgaacgagctagaaccggagaagcagcccatgaatgcagcggacggggcggcggccggggagaagaacggtctggtgaagatcaaggtggcggaggacgagacggaggccggggtcaagttcaccggcttatccaaggaggagctactgaaggtagcgggcagccctggctgggtgcgcacccgctgggcgctgctgctgctcttctggctcggttggctgggcatgctggcgggcgccgtggttatcatcgttcgggcgccgcgctgccgtgagctgcct

SEQ ID NO: 4 - DNA, 4814 bp, synthetic construct
  source 1..4814: mol_type = other DNA
cagtttggtaggctctgcgggtctgtgggactatcctaaaagcagttgcaagccactgtctgactagctccctcaggttggctatgtgctaagatgagtcacaggatcgtttattagtgaaaggacaatgagctgaaggaagaactacataggcagagcctgagggggcagagcctgggggggcagagccggggggggggcagagcctgggggggcagaggggggggtataggaaggtttctcttctcccagtacctgccttagccataaatggtaccaagaagcaggaaactcacttgagccttaggtgaccagagttttggattatctctaagagtttcagtggagccaagaatgaggaatgatgaactgaccctcctgcctctacctgttagaatcacaggcatgcctcaatctgccttccttgttcctgtttcttgttttctttttgaggcagtgtctagcaatgtagtccagactgtcttcaaatttatagcaatctcccctcagtcttctgagtactgggatcacaacgtgcatcaccaagccctctttccttagagtaatcctatcaaccatgtggctaacctagtttccagccctgccagaggctctggagcaatactttaaggccatttcccaaagaggttgaatgctccatgcctcactacggcagcctagttgatacccaccaaacaactcctaagacaaaacctagcagcctagaaatgatcttgaagctaaggacaaaggccagacttacaagcaagagtcattcagaactgtacatagtgtcagaaggagtgcaataggcgttgtgggcctggttttagcaggcttgatgtgtgattagtggaggagaagggaggtagctgaggcaggtttgggactgtgatgaaggatatgtctatggaggaggaagtcagtggaggaggtggtggcatcatgcattgactctgcaggccagttcgcaggatttattgtccttttgtcagccagtgattttttttcttttttttaaagtgggccccataggccttagtgttaactttgccttcaaagtcttaggaacaagaacctggtggatattttggctctcctagccttggggaaaagaacacgttggaccctgtgtgtaggctctacagcaggctggaacttgctgtggcctgggcctctgagggtattctggtgtagaggacagatccaagctactaaaagacctaactaagatccagcctttgtttcttcatcttgctcagtgacagagcctgagcctttgagaggagtccatactgactcccgctctgaagacatacaccgctctccctcccagccccatttaccccctggcttttgtctcttgtggacctcttcctgaggctgcccaccccttccctctgtttactttcctagatcctcccccccatcttccctctctcctcttctttgctgcctagttgtttctttctttttctttctttttctttttctttttctttttctttttctttttctttttctttttctttttcttttttttttttttgtttctgggctaagctctaactatgtagccctggctggaatgcatacagatattcctgcttccccttcccttgtggaaccgccaaagctatcgcccaggggatccattttgacccagattttactgtgaaatattgtggtgttattttctgattattttaaaagttcttttactttttatttttacttttttatgcatgtactttatagattagagaggtgtcttggcaagtaagagcactttctgctcttgcagaagacttgggttcagtttctagttcagtttccaaatggtggctcacccatagctccaggtccaggctctccagtgcgctcttctgaccgctacaggtaccagacacatacgcagtacacaatacataaatgtaggtaaacagtcatacacataacaaaaataaataaatcttaaaaagtttatgttctttggggttggcaagatggctcatcagttaaaagaatgagctactattctagattggtcagatttgagtcccagcacccacatagctgctcgtaatcatctgtaattctagtttcaagagatccagtgcctcttccggcttccaggggaagcagccacacatgtggtgcacagatatacatgcactcaaagcactacacacattaaaaaataataaaaatgccgggtggtggtggcacacacctttaattccaagcacttgggaggcagaggcaggcggatttctgagttcgaggccagcctggtctacaaagtgagttccaggacagccagggctacacagagaaaccctgtctcaaaaaaccaaaacaccaaaaaaaaaaaaaagaaaagaaaaaagtaataataaaaatgtaaacagtagataatagatattaagcaaaccaagatgagacctagttttgctctttacagctaccatggtgcagagaagcctcagaaactgagtttgtctggcagggtcctggcaacagtgcgtctccagatctttgtgttctgggaatataagtgggaacccatcccccacccctgaaatctgcaagttctggaccctggcatatgcaatcgtcttgaaagccaagcacagtctggtctggttagtatttagtaggagaccacctgcgaataccaagttctctaagactaagaatcaaaaatgaaacaaaaggagctgcaggaggtggctcacaactttaatgccagcattccagaggctggggggtgggtgcgggggggggggggggggcagatggatttacataggtcagagagtcaattctctttctaccacattgggtttcagggattgaactcaggtcattaagatggtcaagcaaacgcctttgcctgctgagccatcttcttgccctaagattaaaaatcaactactttctggtgctagggattggatgtagggccttgtgtgtattaggcaagtgctctaccactgggctacattctaagtccttggatggactggatgtaaggggagttgggcagactgggccattaagctaatcagtggaggcaagagaatgaaaggcaggaaggtgatctgcgccttccttggtggtgttctgggcataggttctctagcacctgcacatggagatttctaggagggaagagtacggctcagtacagggcacgatctaggatcacttgaggtatagacagggtgtgtaggaagcattgctgagtgaagagagaggcagcagaggggaggttggagccaaagattgagtgtatttacaggatgaacagtggaagagactctaacaaaggaagagtagaagcaaattgaatggcgggcatgaagagaaggaggcaagaaggtgccaggtagatgttagatagagacagtatctttcagattggtgataaagatattattggctgtctataggggaacttaccaggagccttctggggagaatggaaaagtagttttctttcttgttgcacaggtttctagagatttcaatttgtgtacatggttgtcttaagtaccaaaagaacttattggaaggctatcaggaaatgccagaagaaagcaggatcagactcgggaaatgggcaggaagcaagcaccaagaaagcaggaagttcgattgtcttttaactggaactgcctgggatgctgccacaggggacgcactgccaagatcttgtggttgggcacaaaaagcacaatgcacaaaagcatagcctccctgggtggggtgtgggtggtgttggaatttacaatgtgttctgcttgaatggccctggtatgtcctgagtctcaggactaagagggttattatgtttttaaaatgttctttatttaaaaaaaaaatgaaaatagggtctggagagatgacttagccatgaaacaaaccaggtattttgcacacgcctgtaactccagtttcaaggagagagcaaagaggaggattgttgaggggcggggggggggggggagtgttgttggctttcagctaggaaagaaagcatgagtcccaggttcagaatgagacactgcctcagaggaatagttggagagtgatagaggagagtgttcaaagccctctttcagcctgaacacacagcacacagcaccctcccctcccacacgcacatacacaaataagtaaatatggtttttaaagattttacttattatatgtaaatgagtacactgtagctgtcttcagacacaccagaagagggcatcggatcccattgcagatggttgtgagccaccatgtggttgctggggattgaactcaggacctctggaagagcagtcagttctcttaacctctgagccatctctccagccctaagtaaatattttaaacaatgaaatcagagccagtgagatggctcagtaggtgaatgtacctcctgcagagcctgacaacatgagtttgatccctgagacccacacaacggaaggagagaagctgactcctccaagttgccaggtgacctccatgtatgcacctttgcgtgtgcattgcatcccctgataagtaaatgtaaaaagaaggtcccccacctcacccctttaaagacagggcttctctgtgtagccctggctatcctggaactcaatctgtagaccaggctggcctcgaactcagagcctgcctgcctctgcctcccgagtgccaggattaaagctgtgcccccatgcctagcttctttttcttaaaaactatgattgtgtaggtgtaggctgtgtatgtgtcatgtgcagtcagaggacaactttgtgtca

SEQ ID NO: 5 - DNA, 968 bp, synthetic construct
  source 1..968: mol_type = other DNA
tgatgaagcaaaggattggagtgggtgcccagtaaatagttgctgcctgaggaattaaattcaaggagtcttgaaaggtgagtacgtgtaagccccgaggagaatccaggaactcagctaagtccggagtatgagaagatgtacgtaaagcagaagtgagatgtagtgccaaggtctgtgtgaagttggggagatcagcctgagccgtgcttggggcgaagggatcacgatgtggatggagactggtgtcatcttgtggagagaaaagaggcctcgcagggtaaggggccggaaggtcggaggaggggctttgaaacctggtcaagacaaattcacgtttgttattctaagggtttctgaccacaagctccccttcccagactcttgcaggaataagcgttagggatagacgggagtattcagcgaggctggaggtgcgaaaagacttctctgcatctctttaagaggcggaccggggcggagtcctgggtcacttccccgccgccacacccgcccagcggcagaagcagttaggaagctctgctagcctcacggccacgggacgcctctctgaacggggatccaggcaggattagagctgcctcactgactacaggccgtgtcgtgtcaccgtttctgcaggcaccatgagccaggacaccgaagtggacatgaaagatgtggagctgaacgagctagaaccggagaagcagcccatgaatgcagcggacggggcggcggccggggagaagaacggtctggtgaagatcaaggtggcggaggacgagacggaggccggggtcaagttcaccggcttatccaaggaggagctactgaaggtagcgggcagccctggctgggtgcgcacccgctgggcgctgctgctgctcttctggctcggttggctgggcatgctggcgggcgccgtggttatcatcgttcgggcgccgcgctgccgtgagctgcct

SEQ ID NO: 6 - DNA, 1364 bp, synthetic construct
  source 1..1364: mol_type = other DNA
tccttcctatgcagaacctaccaccctcctttgttctccccaggccttttggattctagtcttcctctccttgtttttaaacttttgcagattacatacgaattcttatactgggtgtttttgtcttcaaataaaaacatcacccctgcctcatgagattgtgactttcatccttccttccttctagaagaactttctcttgctcctgatctcttttgctcctccctgcccctgccatagtcgcagccagttgtagacagctattccagctctctttttttttttttttttttttttttttttggtttttcgagacagggtttctctgtatagccctggctgtcctggaactcactttgtagaccaggctggcctcgaactcagaaatccacctgcctctgcctcccaagtgctgggattaaaggcgtgcgccaccacgcccggccgctattccagctcttaaattaatcatttagagaccaaggctagagaagggcccttccatggttaacagcaaagtgtcttggctggagtaaccacacctcctcgctctggcccaagaatcttgggaattgccaactcttccttatctctcttagcacagtctttaagaaaaagggtggggtgagttgaagactgcatactgccaagggcctggggcttcccttctttactctttggtgaggcacttaccatatagacaggactgcgatccccagtacccagtggataccccatctccagaaaaagccaacaagacaaaccctttgcttccttaggctatgttatctcttgtgtggaaatggagaagaaataaggaataaacattttttgtatgaaggatatggatgagtgatcaaaaaggaaaaggagctggagagatggctcagaggttaagagcactggctgctcttgtagaggctgccaggttcagttcccagcatccacacggctcacaacgatcgtttactcctagtcttgggatcaacacccacttctagttttgcaggcaccaggcttgcatttggtatacaagtataagcacaggcaaagcacacatacataagaaggaaaaagcaaaactagaaagatgtttttgagagcaggtttgggcttgagagcaggagacggttcaaacaagagaggtgagttaactcatgaatgtggcccagtcccaagtcataaacaggttatactgttacagtgtccgaagttagacatggggtgggtctggagaggatattggttatacgaatgagagcatggttgatgctcaggaagcaggagtaaatgagtaaggaacgaaggttattcagcaaggggtgggcagatcacaaagggtttcataaggcagtcagaggtgc

SEQ ID NO: 7 - DNA, 7331 bp, synthetic construct
  source 1..7331: mol_type = other DNA
gcgcagaagtggtggcacacgggcgccctctaccgcatcggcgaccttcaggccttccagggccacggcgcgggcaacctggcgggtgagtgcagcgcgcccccgtcccgggtacctccggttgaatctggtggcttgcaccgaccccctcccctgtccccagacggatctagatggttcttccctccatcccgtaccgacgactgttcccccttcccccaccccctccccggcacattgtccttccctcctttctttgaagaaagccgacccgcccctcactccgtcacgagggtgggtgactcagcgtcctccttccccgcggcgccagaagccagttgcaaccggtttctgaagtaatgtgcaggactccttacatcagctcctctgagtctcgtgattcagccttgcctccctctctccccctttgccccctccccgtcccacccttaggcgctgggagaagggagggtggggaggtcaggggcctctcagaggggcctcacttgttaacccagcccccatttcaggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagtgggtatcctggggttcccaaggaaacagctagaaaggacttggccagaggaaaagtaggctagaacttttgtctggtttcctgctaactggctctgagttttcctagggcaggtagaggggagatttgttagtcttcagcctaaaatggatttgtccagtcgtctgggggctttctacttggctgtggaatcttgagcaggtcatttaacttctgggcttcagtgcccttatttgcaaaatatagatatttactaaaccagtggttttaaacttgtaaaacagtaggtttttttggggggggggaatcccaaatattgttttacgactcgatatagacacatgtttaaaaaggttcactttgggagtggggaacacaggggccacttctctgcactataaactttctgccttccaatatctttttttttttttgagatggagtctcactcttgtcacccaggctgcagtgcagtggaacgatctcagcttactgcagcctcctcccccagggttcaagcgattctcgtgcctcagcctcctgagtagctggtactacagccgtgtgccaccacacccagataatttttttatttttagtagagacaattttcaacatgttgaccggtctggttttgaactcgcctggcctgccttcccatatcttaaggcccttccttgctcacatctgtttcaatggaatgaattttgcccgggttcaaattcaagcttttgtaccaagtaatacataggaaacactcagtgacttgcatttgtgatttccttattttcccttaactcatagactgtctcatgattgcgtcttcctccgttgttttaggcatccgtgtcattctggaccttactcccaactaccggggtgagaactcgtggttctccactcaggttgacactgtggccaccaaggtgaaggtgagtgttggagctgatggctggtggaagtcagatgctggggctgggacagtcctttcacagcaagccctgtagacccagcctgactccagctgagggtccctttgctccttaggaccaaggaggaatgcctcctcctatagccaaaggataggccaatagcacacctgtttttcttgttccttttctgacctcttatttttcttatttccttgactctgaggatgagacagtgccaggaagggaccttctcagaagtcagtctggaagtttgatttttctttcttttgggagaggaagccctttctctgtgagaatcctgtcctgactggtttcccagttcttgtttgatgtgtttggaatagagcccagtctgaaattataagcactggggtactcctatgtgtctgggactctatgccaagtattttatagatgttttctaattctctgtataaccatacaaggtatataatttgccataggttcaaatgtgtacccagcaggctaggagtagggctttgaatgtcacctagctcctgatgtctgggtcctaggacactgacctgttcagaatgggtagaacaaaaatggaagcagatactgacactgctggtagctgtgattctaagatagaattctggactcccagcatttgggtaggaccccgggatcatccagtttaggccttgccagttactaggggaccctttaaaagttgctgatgagggaatgtttttggatggtttggcatgaggggcggtaccctcagatggaaatggagaccagaggagagagtacatggcccagagaaagggaagaaaaggagaatgggggtgaagttccacagtgtgtgcaggactgccaggattccattcagcagcactggcctcttttcaaatctaaactgccctggggctatgctttgaatttcaatctttcatccttggccttgctctgagcagttattgtactcacaccttccccaggcctcaggaaactgggagaggggggatcttgactgacattttagatcctgtctgctgctcactgattatgactgtggacgcgtctccctgcttctctgggtctgtttcttcatctctaaaataggtatgctaacattccaatgctaagtgttaaacgaagttatgtttatggacataccttttctctggggccttttctgtggctttcaaagttgaagctgtgtgatttgccagaggcaagaatgagaagtaaaatgctgtagcactaagaaaatgagtggaagggtagagctggaggggtccaaaggctaagcctttgtagtgcaggtggggaagacccaggcaaggcacaggactctccccagctcccggggaagtgtgtggtgggtgaggtttagtgtgggctggaattttctgagccttttcctgagaactgatgtctgtcctttattcttctgccccctataggatgctctggagttttggctgcaagctggcgtggatgggttccaggttcgggacatagagaatctgaaggtgagttccctttccacattagggacaaagcttgggcgagaacagagggactcagctagagcctcataatattctctggtccttgattctgcttttttctttctaggatgcatcctcattcttggctgagtggcaaaatatcaccaagggcttcagtgaagacaggtgggtgcaggagccattctgctgactcagctccaatgtgggaacccctcagtggagtgctaggcctaagaaggggggattcctagtctagagcattttctttacctttattctttctttagctttttttttttttttttttttttttttttttttttgagatggagttccgctctttttgcccaggctggggtgcgatggcgtgatcttggctcactgcaacctctgcctcctgggttcaagcgattctcctgcctcagcatcccaagtagctgggattacagacgcgggccaccacacctggctaattttgtatttttagtagagacggggtttctccatgttggtcaagccagtctcgaactcctgacctcaagtgatccacccgcctcagcctcccaaagtgttgggattacaggcgtgagccactgcgcctggccccattctttcttgtgctaaccttgaactcctccctcccctctgcaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtgtgagtaccatgctggtgggaaagggggcagatgggagaagaaagggttgttgggagacagaggcagaggtgggttatggggctcactggagtgtctctccctgtagttgtctcaggcaaggctcctgacttccttcttgccggctcaacttctccgactctaccagctgatgctcttcaccctgccagggacccctgttttcagctacggggatgagattggcctggatgcagctgcccttcctggacaggtactgcttgctgtctttctgtcacagggaggttgtttatccatcttacaatgatgattctggggatggcggcacatagacgtgagccttggggtgaaaacgcgtttgattcttagtcagtagctgagtggctatgtggctttgggcaaattattttacctttctgaattttggtcatcccttgtgttactgcatttcttgaagggtggttagtattaaatgagggcatgtttttaatggtaatgcctggtacctggcagatgtttgctatggtagccaccattcatttattcattcctttattctttggacagtgggtactgtgctaggctctgtgccctcaggagctcatagtttagtgagggagccagataaataagtggagaagtacatggcagtggctgggcacggtggctcatgcctgtaatcctagcactttgggagtccaaggtgggtggatcactggaggtaaggagttcgaaaccagcctagccaacagggtgaaacccagtctctactaaaaatacaaaaattagccaggtgtggtggtgcacgcctgtagtcccagctactggggaggctgaggcaagagaatcgcttgaacctggaggcagaggttgcagtgaactgagattgtgccactgcactttagcctgggcggcagagggagactccttctccaaaaaaaaaaagaaaagaaaagaaagaaagaaatacatggtagggatactactacaaaggcattcactagctttagatacagggttactgttgatccccgactttttttttttgtttgtttgtttgttttttgtttttgtttttaagacagcatttccctatgtcgcccaggctggagtgcagtggcatgatcttggttcattgcagcctccgcctcccaggccgaagtgatcatcccacctcagcctcctgagtagctgggatatgtaccatagataggtgcgtgccatcatgcccagctgatcttttttttttttggagacaggttcttgctctgttgatctggctggattgcagtggtgcgattatgtctcactgcaacctctgcctcttgggttcaagtgatactcctgcctcagcttcccaagtagctgggaatacaggtggtctgggaatacaggcacacaccaccatgcccggctaatttttgtagagacaggctttcactgttgttgcccaggctggtctccaactcctgggctcaatcgctctgcctgccctgatctcccaaattgctgggactacaggtgtgagccactgtgcctggccacagctaatttttaaaatttgtagaggtgtggtctcactatgttgcccaggctgttctcaaactcctgggtgcaaatgatcctcccaccttggcctcccaaactgctatgattacaggtgcgagccactgcacccggccatcagcatgatgtttatagaaccagttattcatttattccacagatgtttatagaataatttctgtctgttacactgaggatacagcattgcacaagagagctggaggtggttagataattagatatgcataaataaaacgccagctgctgatcagtgctatgcaaaagaaggcggggtaaggagcctagagactaggggatgatggggcttgttttatagagagtagccataggagacctggtaagataaccaagcagatacctgagggaagtgagagagctggctggcagccatctgggagctcttggctgaggggcaacagtgtatgtgtgaggaccctgaggtgggagcctgcctcactcatccagtagaagttagccaggccgggtgtcgtggcttatgcctgtaatcccagcactttgggaggccaaggtgggtggatcacctgaggtcaagagttcgagaccagcctagccaacatggtgaaaccccgtctttactaaaaatacaaaaaaattagctgggtgtggtggtgcacacctatggtcagctactggggaggctgaggcaggagaatcgcttgaacccaggaggcagagattgcagtaagccaagatcgtgccattgcactccagcctgaacagagcgagactccgtctcaaaaaaaaaaaaaaaaaaaagttagccagacatgagttggggaggttatatattgatgaggatgtgaatgggaagagggatacgggacagtgggcccagggctttttttttgagacagggtctcactctgttgcccaggctggagtacagtggtggagtcatggctcactgcaagctcaaactcctgggttcaagtgatcctccctcctcagcctccccagtatctgggactatagggatgtaacactacacccagctaatttttaaaaattttgtagtagagatgagggctcattatgttgctgaggctggtttcaaatgcctgggctcaagcaatcctcttggccttgacctcccagactgctggaattacagatgtgagccaccatgcctgaccccaggcctttttaaagtcctattttctctgcttttcagcctatggaggctccagtcatgctgtgggatgagtccagcttccctgacatcccaggggctgtaagtgccaacatgactgtgaaggtaagagttctagatgggtagaaactgaccggtggagggtgggctgggcttgtagaagtctgtacccaggggagcccctcacacgcttctgctatgtttttatcattcttagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctga

SEQ ID NO: 8 - DNA, 80 bp, synthetic construct
  source 1..80: mol_type = other DNA
ggcagatcacaaagggtttcataaggcagtcagaggtgcaaagcttcctagggatatcgaattccgaagttcctattctc

SEQ ID NO: 9 - DNA, 80 bp, synthetic construct
  source 1..80: mol_type = other DNA
gtataggaacttcatcagtcaggtacataatggtggatcccagtttggtaggctctgcgggtctgtgggactatcctaaa

SEQ ID NO: 10 - RNA, 2804 nt, synthetic construct
  source 1..2804: mol_type = other RNA
gtgggtagaggaatccgcccaaaggggcgtgcggagagctccgcctctgattttgcagcgcgaaaaagaggcgcaggcgctttaggggagtgcgacgctacgcctttggcgctgcggctaggcggttcttactcactgcgggtaaaacgtcatcgctggagattttggttcgcgacccatacagctcgactgtctgggtcacaactaccaatatccatacgttgaggcgatttctcaccctcactcacgctaagccgcgtgttgatccatctctatggatcctgaacctactgaacactccaccgacggtgtctcggttccccgccagccgcccagcgcgcagacggggcttgatgtccaggttgtcagcgcagcgggcgactcaggcaccatgagccaggacaccgaagtggacatgaaagatgtggagctgaacgagctagaaccggagaagcagcccatgaatgcagcggacggggcggcggccggggagaagaacggtctggtgaagatcaaggtggcggaggacgagacggaggccggggtcaagttcaccggcttatccaaggaggagctactgaaggtagcgggcagccctggctgggtgcgcacccgctgggcgctgctgctgctcttctggctcggttggctgggcatgctggcgggcgccgtggttatcatcgttcgggcgccgcgctgccgtgagctgcctgcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgatccttcctatgcagaacctaccaccctcctttgttctccccaggccttttggattctagtcttcctctccttgtttttaaacttttgcagattacatacgaattcttatactgggtgtttttgtcttcaaataaaaacatcacccctgcctcatgagattgtgactttcatccttccttccttctagaagaactttctcttgctcctgatctcttttgctcctccctgcccctgccatagtcgcagccagttgtagacagctattccagctctctttttttttttttttttttttttttttttggtttttcgagacagggtttctctgtatagccctggctgtcctggaactcactttgtagaccaggctggcctcgaactcagaaatccacctgcctctgcctcccaagtgctgggattaaaggcgtgcgccaccacgcccggccgctattccagctcttaaattaatcatttagagaccaaggctagagaagggcccttccatggttaacagcaaagtgtcttggctggagtaaccacacctcctcgctctggcccaagaatcttgggaattgccaactcttccttatctctcttagcacagtctttaagaaaaagggtggggtgagttgaagactgcatactgccaagggcctggggcttcccttctttactctttggtgaggcacttaccatatagacaggactgcgatccccagtacccagtggataccccatctccagaaaaagccaacaagacaaaccctttgcttccttaggctatgttatctcttgtgtggaaatggagaagaaataaggaataaacattttttgtatgaag

SEQ ID NO: 11 - protein, 562 aa, synthetic construct
MDPEPTEHSTDGVSVPRQPPSAQTGLDVQVVSAAGDSGTMSQDTEVDMKDVELNELEPEKQPMNAADGAAAGEKNGLVKIKVAEDETEAGVKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 12 - DNA, 23 bp, synthetic construct
  source 1..23: mol_type = other DNA
tttgtaggccgggatgcgggagg

SEQ ID NO: 13 - DNA, 23 bp, synthetic construct
  source 1..23: mol_type = other DNA
actaagcaaaagtttagcgctgg

SEQ ID NO: 14 - DNA, 132 bp, synthetic construct
  source 1..132: mol_type = other DNA
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 15 - DNA, 25 bp, synthetic construct
  source 1..25: mol_type = other DNA
aatctcttctggatgcctgattccc

SEQ ID NO: 16 - DNA, 22 bp, synthetic construct
  source 1..22: mol_type = other DNA
gttgcagtgagccaagatcacg

SEQ ID NO: 17 - DNA, 25 bp, synthetic construct
  source 1..25: mol_type = other DNA
cattagggacaaagcttgggcgaga

SEQ ID NO: 18 - DNA, 24 bp, synthetic construct
  source 1..24: mol_type = other DNA
cctcaggctctgcctatgtagttc

SEQ ID NO: 19 - DNA, 27 bp, synthetic construct
  source 1..27: mol_type = other DNA
acatctggttggtttgatccatcttgt

SEQ ID NO: 20 - DNA, 25 bp, synthetic construct
  source 1..25: mol_type = other DNA
ggccagaacagtagagaggaaggga

SEQ ID NO: 21 - DNA, 22 bp, synthetic construct
  source 1..22: mol_type = other DNA
cactgcacccggccatcagcat

SEQ ID NO: 22 - DNA, 26 bp, synthetic construct
  source 1..26: mol_type = other DNA
ctcacacatacactgttgcccctcag

SEQ ID NO: 23 - DNA, 20 bp, synthetic construct
  source 1..20: mol_type = other DNA
aggtgaagggcctggtgtta

SEQ ID NO: 24 - DNA, 20 bp, synthetic construct
  source 1..20: mol_type = other DNA
atgctggactcattccacgg

SEQ ID NO: 25 - DNA, 20 bp, synthetic construct
  source 1..20: mol_type = other DNA
ctgaaggggcgtctcgatta

SEQ ID NO: 26 - DNA, 20 bp, synthetic construct
  source 1..20: mol_type = other DNA
catcccacagcatgactgga

SEQ ID NO: 27 - DNA, 22 bp, synthetic construct
  source 1..22: mol_type = other DNA
tcaccatcttccaggagcgaga

SEQ ID NO: 28 - DNA, 21 bp, synthetic construct
  source 1..21: mol_type = other DNA
gaaggccatgccagtgagctt

SEQ ID NO: 29 - protein, 566 aa, Rattus norvegicus
MDPEPTEHSTGGGSVPRQPPSAQTGLDVQVVSAAGDSGTMSQDTEVDMKDVELNELEPEKQPMNAADGAAAGEKNGLVKIKVAEDEAEAGVKFTGLSKEELLKVAGSPGWVRTRWALLLLFWLGWLGMLAGAVVIIVRAPRCRELPVQRWWHKGALYRIGDLQAFVGPEARGIAGLKNHLEYLSTLKVKGLVLGPIHKNQKDEVNETDLKQIDPDLGSQEDFKDLLQSAKKKSIHIILDLTPNYKGQNAWFLPPQADIVATKMKEALSSWLQDGVDGFQVRDVGKLANASLYLAEWQNITKNFSEDRLLIAGTASSDLQQIVNILESTSDLLLTSSYLSQPVFTGEHAELLVIKYLNATGSRWCSWSVSQAGLLTSFIPAQFLRLYQLLLFTLPGTPVFSYGDELGLQAVALPGQPMEAPFMLWNESSNSQTSSPVSLNMTVKGQNEDPGSLLTQFRRLSDLRGKERSLLHGDFDALSSSSGLFSYVRHWDQNERYLVVLNFQDVGLSARVGASNLPAGISLPASANLLLSTDSTRLSREEGTSLSLENLSLNPYEGLLLQFPFVA

(19) United States
(12) Patent Application Publication
(10) Pub. No.: US 2026/0123609 A1
(43) Pub. Date: May 7, 2026

(54) GENETICALLY MODIFIED NON-HUMAN ANIMAL WITH HUMAN OR CHIMERIC CD98HC

(71) Applicants: Biocytogen JiangSu Co., Ltd., Nantong, Jiangsu (CN); Biocytogen Pharmaceuticals (Beijing) Co., Ltd., Beijing (CN)

(72) Inventors: Ruili Lv, Beijing (CN); Zhiyuan Shen, Beijing (CN); Chong Li, Beijing (CN)

(21) Appl. No.: 18/704,232

(22) PCT Filed: Dec. 16, 2022
(86) PCT No.: PCT/CN2022/139671

§ 371 Date: Apr. 24, 2024

(30) Foreign Application Priority Data
Dec. 17, 2021 (CN) 202111550049.0

(51) Int. Cl. A01K 67/0278; C07K 14/705; C07K 16/28; C12N 15/90

(52) U.S. Cl. CPC A01K 67/0278; C07K 14/70596; C07K 16/2896; C12N 15/907; A01K 2207/15; A01K 2217/072; A01K 2227/105; A01K 2267/0331

(57) ABSTRACT

Provided are genetically modified non-human animals that express a human or chimeric (e.g., humanized) CD98HC, and methods of use thereof.

### CLAIM OF PRIORITY

This application claims the benefit of Chinese Patent Application App. No. 202111550049.0, filed on Dec. 17, 2021. The entire contents of the foregoing application are incorporated herein by reference.

### TECHNICAL FIELD

This disclosure relates to genetically modified animal expressing human or chimeric (e.g., humanized) CD98HC, and methods of use thereof.

### BACKGROUND

The traditional drug research and development typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results.

Therefore, the development of humanized animal models that are suitable for human antibody screening and evaluation will significantly improve the efficiency of new drug development and reduce the cost for drug research and development.

### SUMMARY

This disclosure is related to an animal model with human CD98HC or chimeric CD98HC. The animal model can express human CD98HC or chimeric CD98HC (e.g., humanized CD98HC) protein in its body. It can be used in the studies on the function of CD98HC gene, and can be used in the screening and evaluation of anti-human CD98HC antibodies or drugs targeting CD98HC. In addition, the animal models prepared by the methods described herein can be used in drug screening, pharmacodynamics studies, treatments for immune-related diseases, and cancer therapy for human CD98HC target sites; they can also be used to facilitate the development and design of new drugs, and save time and cost. In summary, this disclosure provides a powerful tool for studying the function of CD98HC protein and a platform for screening cancer drugs.

In one aspect, the disclosure is related to a genetically-modified, non-human animal whose genome comprises at least one chromosome comprising a sequence encoding a human or chimeric cluster of differentiation 98 heavy chain (CD98HC).

In some embodiments, the sequence encoding the human or chimeric CD98HC is operably linked to an endogenous regulatory element at the endogenous CD98HC gene locus in the at least one chromosome.

In some embodiments, the sequence encoding a human or chimeric CD98HC comprises a sequence encoding an amino acid sequence that is at least 70%, 75%, 80%, 85%, 90%, 95%, 99%, or 100% identical to human CD98HC (NP_002385.3 (SEQ ID NO: 2)).

In some embodiments, the sequence encoding a human or chimeric CD98HC comprises a sequence encoding an amino acid sequence that is at least 70%, 75%, 80%, 85%, 90%, 95%, 99%, or 100% identical to SEQ ID NO: 11.

In some embodiments, the sequence encoding a human or chimeric CD98HC comprises a sequence encoding an amino acid sequence that is at least 70%, 75%, 80%, 85%, 90%, 95%, 99%, or 100% identical to amino acids 215-630 of SEQ ID NO: 2.

In some embodiments, the animal is a mammal, e.g., a monkey, a rodent, a mouse, or a rat.

In some embodiments, the animal is a mouse.

In some embodiments, the animal does not express endogenous CD98HC or expresses a decreased level of endogenous CD98HC as compared to CD98HC expression level in a wild-type animal.

In some embodiments, the animal has one or more cells expressing human or chimeric CD98HC.

In some embodiments, the animal has one or more cells expressing human or chimeric CD98HC, and the expressed human or chimeric CD98HC can interact with a human integrin, activating downstream signaling pathways.

In some embodiments, the animal has one or more cells expressing human or chimeric CD98HC, and the expressed human or chimeric CD98HC can interact with an endogenous integrin, activating downstream signaling pathways.

In one aspect, the disclosure is related to a genetically-modified, non-human animal, wherein the genome of the animal comprises a replacement of a sequence encoding a region of endogenous CD98HC with a sequence encoding a corresponding region of human CD98HC at an endogenous CD98HC gene locus.

In some embodiments, the sequence encoding the corresponding region of human CD98HC is operably linked to an endogenous regulatory element at the endogenous CD98HC locus, and one or more cells of the animal expresses a human or chimeric CD98HC.

In some embodiments, the animal does not express endogenous CD98HC or expresses a decreased level of endogenous CD98HC as compared to CD98HC expression level in a wild-type animal.

In some embodiments, the replaced sequence encodes all or a portion of the extracellular region of CD98HC.

In some embodiments, the animal has one or more cells expressing a chimeric CD98HC having an extracellular region, a transmembrane region, and a cytoplasmic region, wherein the extracellular region comprises a sequence that is at least 50%, 60%, 70%, 80%, 90%, 95%, or 99% identical to the extracellular region of human CD98HC (NP_002385.3 (SEQ ID NO: 2)).

In some embodiments, the extracellular region of the chimeric CD98HC has a sequence that has at least 10, 20, 30, 40, 50, 60, 70, 80, 90, 100, 110, 120, 130, 140, 150, 160, 170, 180, 190, 200, 250, 300, 350, 400, 410, or 416 contiguous amino acids that are identical to a contiguous sequence present in the extracellular region of human CD98HC (e.g., amino acids 215-630 of SEQ ID NO: 2).

In some embodiments, the chimeric CD98HC has a sequence that is at least 10, 20, 30, 40, 50, 60, 70, 80, 90, 100, 110, 111, 112, 113, or 114 contiguous amino acids that are identical to a contiguous sequence present in the cytoplasmic region of endogenous CD98HC (e.g., amino acids 1-114 of SEQ ID NO: 1).

In some embodiments, the sequence encoding a region of endogenous CD98HC comprises exon 1, exon 2, exon 3, exon 4, exon 5, exon 6, exon 7, exon 8, exon 9, and/or exon 10, or a part thereof, of the endogenous CD98HC gene.

In some embodiments, the animal is a mouse.

In some embodiments, the animal is heterozygous with respect to the replacement at the endogenous CD98HC gene locus.

In some embodiments, the animal is homozygous with respect to the replacement at the endogenous CD98HC gene locus.

In one aspect, the disclosure is related to a method for making a genetically-modified, non-human animal, comprising: replacing in at least one cell of the animal, at an endogenous CD98HC gene locus, a sequence encoding a region of endogenous CD98HC with a sequence encoding a corresponding region of human CD98HC.

In some embodiments, the sequence encoding the corresponding region of human CD98HC comprises exon 1, exon 2, exon 3, exon 4, exon 5, exon 6, exon 7, exon 8, exon 9, exon 10, exon 11, and/or exon 12, or a part thereof, of a human CD98HC gene.

In some embodiments, the sequence encoding the corresponding region of human CD98HC comprises a portion of exon 4 (e.g., at least 50 bp), exons 5-11, and a portion of exon 12 (e.g., at least 200 bp), of a human CD98HC gene.

In some embodiments, the sequence encoding the corresponding region of human CD98HC encodes amino acids 215-630 of SEQ ID NO: 2.

In some embodiments, the region comprises all or a portion of the extracellular region of CD98HC.

In some embodiments, the sequence encoding a region of endogenous CD98HC comprises exon 1, exon 2, exon 3, exon 4, exon 5, exon 6, exon 7, exon 8, exon 9, and/or exon 10, or a part thereof, of the endogenous CD98HC gene.

In some embodiments, the animal is a mouse, and the sequence encoding a region of endogenous CD98HC comprises a portion of exon 2 (e.g., at least 50 bp), exons 3-9, and a portion of exon 10 (e.g., at least 200 bp) of the endogenous CD98HC gene.

In one aspect, the disclosure is related to a non-human animal comprising at least one cell comprising a nucleotide sequence encoding a humanized CD98HC polypeptide, wherein the humanized CD98HC polypeptide comprises at least 200 contiguous amino acid residues that are identical to the corresponding contiguous amino acid sequence of a human CD98HC extracellular region, wherein the animal expresses the humanized CD98HC polypeptide.

In some embodiments, the humanized CD98HC polypeptide has at least 200, 250, 300, 350, 400, 410, or 416 contiguous amino acid residues that are identical to the corresponding contiguous amino acid sequence of human CD98HC extracellular region (e.g., amino acids 215-630 of SEQ ID NO: 2).

In some embodiments, the humanized CD98HC polypeptide has at least 10, 20, 30, 40, 50, 60, 70, 80, 90, 100, 110, 120, 130, 140, or 146 contiguous amino acid residues that are identical to the contiguous amino acid sequence of endogenous CD98HC (e.g., amino acids 1-146 of SEQ ID NO: 1).

In some embodiments, the humanized CD98HC polypeptide comprises a sequence that is at least 90%, 95%, or 99% identical to amino acids 215-630 of SEQ ID NO: 2.

In some embodiments, the nucleotide sequence is operably linked to an endogenous CD98HC regulatory element of the animal.

In some embodiments, the chimeric CD98HC polypeptide comprises an endogenous CD98HC transmembrane region and/or an endogenous CD98HC cytoplasmic region.

In some embodiments, the nucleotide sequence is integrated to an endogenous CD98HC gene locus of the animal.

In some embodiments, the humanized CD98HC polypeptide has at least one mouse CD98HC activity and/or at least one human CD98HC activity.

In one aspect, the disclosure is related to a method of making a genetically-modified animal cell that expresses a chimeric CD98HC, the method comprising: replacing at an endogenous CD98HC gene locus, a nucleotide sequence encoding a region of endogenous CD98HC with a nucleotide sequence encoding a corresponding region of human CD98HC, thereby generating a genetically-modified animal cell that includes a nucleotide sequence that encodes the chimeric CD98HC, wherein the animal cell expresses the chimeric CD98HC.

In some embodiments, the animal is a mouse.

In some embodiments, the chimeric CD98HC comprises a human or humanized CD98HC extracellular region; and a transmembrane and/or a cytoplasmic region of mouse CD98HC.

In some embodiments, the chimeric CD98HC comprises an amino acid sequence that is at least 50%, 60%, 70%, 80%, 90%, 95%, or 99% identical to the amino acid sequence set forth in SEQ ID NO: 11.

In some embodiments, the nucleotide sequence encoding the chimeric CD98HC is operably linked to an endogenous CD98HC regulatory region, e.g., promoter.

In some embodiments, the animal further comprises a sequence encoding an additional human or chimeric protein.

In some embodiments, the additional human or chimeric protein is selected from the group consisting of: programmed cell death protein 1 (PD-1), programmed cell death ligand 1 (PD-L1), lymphocyte-activation gene 3 (LAG3), tumor necrosis factor receptor superfamily member 9 (4-1BB), CD40, cytotoxic T-lymphocyte-associated protein 4 (CTLA4), CD147, Interleukin 6 receptor (IL6R), Interleukin 17 (IL17), CD3, CD28, and CD38.

In one aspect, the disclosure is related to a method of determining effectiveness of a therapeutic agent for the treatment of cancer, comprising: administering the therapeutic agent to the animal described herein, wherein the animal has a tumor; and determining inhibitory effects of the therapeutic agent to the tumor.

In some embodiments, the therapeutic agent is an anti-CD98HC antibody.

In some embodiments, the tumor comprises one or more cancer cells that are injected into the animal.

In some embodiments, determining inhibitory effects of the anti-CD98HC antibody to the tumor involves measuring the tumor volume in the animal.

In some embodiments, the cancer is lymphoma, bladder cancer, breast cancer, lung cancer, squamous cell carcinoma, intestinal adenoma, or leukemia.

In one aspect, the disclosure is related to a method of determining effectiveness of an anti-CD98HC antibody and an additional therapeutic agent for the treatment of cancer, comprising administering the anti-CD98HC antibody and the additional therapeutic agent to the animal described herein, wherein the animal has a tumor; and determining inhibitory effects on the tumor.

In some embodiments, the animal further comprises a sequence encoding a human or chimeric PD-1, a human or chimeric PD-L1, and/or a human or chimeric CTLA4.

In some embodiments, the additional therapeutic agent is an anti-PD-1 antibody, an anti-PD-L1 antibody, or an anti-CTLA4 antibody.

In some embodiments, the tumor comprises one or more tumor cells that express PD-L1.

In some embodiments, the tumor comprises one or more cancer cells that are injected into the animal.

In some embodiments, determining inhibitory effects of the treatment involves measuring the tumor volume in the animal.

In some embodiments, the animal has lymphoma, bladder cancer, breast cancer, lung cancer, squamous cell carcinoma, intestinal adenoma, or leukemia.

In one aspect, the disclosure is related to a method of determining effectiveness of a therapeutic agent for treatment an immune disorder, comprising: a) administering the therapeutic agent to the animal described herein, wherein the animal has the immune disorder; and b) determining effects of the therapeutic agent to the immune disorder.

In one aspect, the disclosure is related to a method of determining effectiveness of a therapeutic agent for reducing an inflammation, comprising: a) administering the therapeutic agent to the animal described herein, wherein the animal has the inflammation; and b) determining effects of the therapeutic agent to the inflammation.

In one aspect, the disclosure is related to a method of determining effectiveness of a therapeutic agent for treating an autoimmune disease, comprising: a) administering the therapeutic agent to the animal described herein, wherein the animal has the autoimmune disease; and b) determining effects of the therapeutic agent to the autoimmune disease.

In some embodiments, the autoimmune disease is type 1 diabetes or multiple sclerosis.

In one aspect, the disclosure is related to a method of determining toxicity of a therapeutic agent comprising: a) administering the therapeutic agent to the animal described herein; and b) determining effects of the therapeutic agent to the animal.

In some embodiments, the therapeutic agent is an anti-CD98HC antibody.

In some embodiments, determining effects of the therapeutic agent to the animal involves measuring the body weight, red blood cell count, hematocrit, and/or hemoglobin of the animal.

In one aspect, the disclosure is related to a protein comprising an amino acid sequence, wherein the amino acid sequence is one of the following:
an amino acid sequence set forth in SEQ ID NO: 1, 2, or 11;
an amino acid sequence that is at least 90% identical to SEQ ID NO: 1, 2, or 11;
an amino acid sequence that is at least 91%, 92%, 93%, 94%, 95%, 96%, 97%, 98%, or 99% identical to SEQ ID NO: 1, 2, or 11;
an amino acid sequence that is different from the amino acid sequence set forth in SEQ ID NO: 1, 2, or 11 by no more than 10, 9, 8, 7, 6, 5, 4, 3, 2 or 1 amino acid; and
an amino acid sequence that comprises a substitution, a deletion and/or insertion of one, two, three, four, five or more amino acids to the amino acid sequence set forth in SEQ ID NO: 1, 2, or 11.

In one aspect, the disclosure is related to a nucleic acid comprising a nucleotide sequence, wherein the nucleotide sequence is one of the following:
a sequence that encodes the protein described herein; SEQ ID NO: 3, 4, 5, 6, 7, 8, 9, 10, 12, or 13;
a sequence that is at least 90% identical to SEQ ID NO: 3, 4, 5, 6, 7, 8, 9, 10, 12, or 13; and
a sequence that is at least 91%, 92%, 93%, 94%, 95%, 96%, 97%, 98%, or 99% identical to SEQ ID NO: 3, 4, 5, 6, 7, 8, 9, 10, 12, or 13.

In one aspect, the disclosure is related to a cell comprising the protein described herein and/or the nucleic acid described herein.

In one aspect, the disclosure is related to an animal comprising the protein described herein and/or the nucleic acid described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human CD98HC gene loci.

FIG. 2 is a schematic diagram showing humanized CD98HC gene locus.

FIG. 3 is a schematic diagram showing a CD98HC gene targeting strategy (targeting vector V1).

FIG. 4 is a schematic diagram showing a CD98HC gene targeting strategy (targeting vector V2).

FIGS. 5A-5B show PCR identification results of F0 generation mice. M is a marker. WT is a wild-type control. H₂O is a water control.

FIGS. 6A-6B show PCR identification results of F1 generation mice. M is a marker. WT is a wild-type control. H₂O is a water control.

FIG. 7 shows Southern Blot results. WT is a wild-type control.

FIGS. 8A-8C show RT-PCR detection results of mouse CD98HC mRNA, humanized CD98HC mRNA, and GAPDH mRNA, respectively, in the kidney tissues of a wild-type C57BL/6 mouse (+/+) and a CD98HC gene humanized homozygous mouse (H/H). H₂O is a water control.

FIGS. 9A-9D shows exemplary immunofluorescence (IF) staining results regarding protein expression in the brain of C57BL/6 wild-type mice.

FIGS. 10A-10D shows exemplary immunofluorescence (IF) staining results regarding protein expression in the brain of CD98HC humanized homozygous mice.

FIG. 11 shows the alignment between human CD98HC amino acid sequence (NP_002385.3; SEQ ID NO: 2) and mouse CD98HC amino acid sequence (NP_001154885.1; SEQ ID NO: 1).

FIG. 12 shows the alignment between human CD98HC amino acid sequence (NP_002385.3; SEQ ID NO: 2) and rat CD98HC amino acid sequence (NP_062156.2; SEQ ID NO: 29).

### DETAILED DESCRIPTION

This disclosure relates to transgenic non-human animal with human or chimeric (e.g., humanized) CD98HC, and methods of use thereof.

Experimental animal models are an indispensable research tool for studying the effects of drugs targeting CD98HC (e.g., anti-CD98HC antibodies). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

### CD98HC

The eukaryotic protein CD98hc (also known as 4F2, FRP-1, or SLC3A2) is a membrane glycoprotein and one of the heavy chains of the family of heterodimeric amino acids transporters. The CD98 heterodimer consists of a type II single-pass transmembrane heavy chain (CD98hc, also known as 4F2 antigen heavy chain or FRP-1; encoded by the genes SLC3A2 and Slc3a2 for human and mouse, respectively) of ˜80-85 kDa that is disulfide-linked with a multi-pass light chain of ˜40 kDa.

CD98hc binds to cytoplasmic tails of integrin-β chains and mediates adhesive signals that control cell spreading, survival and growth. The CD98 light chain can be any one of six permease-type amino acid transporters and is bound to CD98hc by disulfide bond. The light chain functions in amino acid transport. Through this nutrient function, CD98 can contribute to the survival and growth of many cell types. CD98hc is also involved in mediation of intracellular integrin signaling. Thus, CD98hc functions in amplifying integrin signaling and in the transport of amino acids; both of these functions can contribute to cell survival and proliferation.

CD98 is implicated in a large number of cancers. Overexpression of CD98hc and CD98 light chains is seen in a large number of cancers, and is associated with aggressive disease. These include lymphoma, leukemia, and lung cancer. Many tumors express CD98hc, and its expression correlates with poor prognosis in B cell lymphomas. Furthermore, the expression of CD98hc or CD98 light chains in solid tumors is correlated with progressive or metastatic tumors. CD98 promotes transformation and tumor growth. CD98 overexpression drives both anchorage independence and tumorigenesis, and the degree of transformation correlates with the level of CD98hc present in the cells. The transforming ability of CD98hc is lost in a mutant that ablates the ability to form disulfide bonds with the light chain and thus abolishes its amino acid transport function. The CD98 domain that is involved in integrin signaling is required for transforming Chinese hamster ovary (CHO) cells and for the development of embryonic stem cell teratomas in mice. CD98 inhibitors have resulted in the inhibition of cellular proliferation and tumor growth both in vitro and in vivo. Anti-CD98 antibodies specific for the heavy chain inhibited the growth of human tumor cells in vitro.

CD98hc might also contribute to other pathologies. Loss of T cell CD98 prevents disease development in two models of T-cell-mediated autoimmunity, type 1 diabetes (T1D) and multiple sclerosis. This raises the possibility that CD98hc could serve as a therapeutic target for blocking inappropriate adaptive immune responses. In addition to autoimmunity disorders, CD98hc overexpression could lead to other adaptive immunopathologies. Arterial restenosis and intestinal villous inflammation are dependent on CD98hc in animal models.

A detailed description of CD98HC and its function can be found, e.g., in Vection, Sonia, David O'Callaghan, and Anne Keriel. “CD98hc in host-pathogen interactions: roles of the multifunctional host protein during infections.” FEMS Microbiology Reviews (2022); Cantor, Joseph M., and Mark H. Ginsberg. “CD98 at the crossroads of adaptive immunity and cancer.” Journal of cell science 125.6 (2012): 1373-1382; Ip, Hugh, and Tariq Sethi. “CD98 signals controlling tumorigenesis.” The international journal of biochemistry & cell biology 81 (2016): 148-150; each of which is incorporated by reference in its entirety.

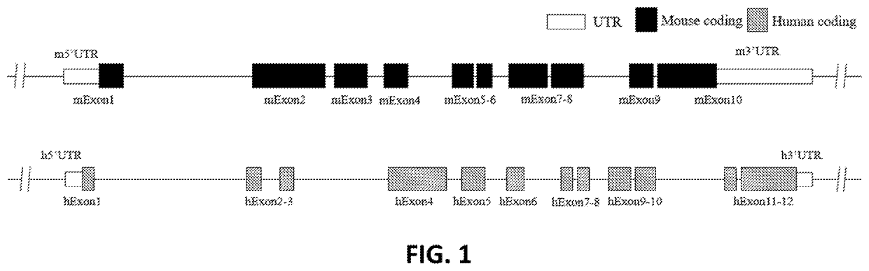

In human genomes, CD98HC gene (Gene ID: 6520) locus has 12 exons, exon 1, exon 2, exon 3, exon 4, exon 5, exon 6, exon 7, exon 8, exon 9, exon 10, exon 11, and exon 12 (FIG. 1). The CD98HC protein also has, from N-terminus to C-terminus, an extracellular region, a transmembrane region, and a cytoplasmic region. The nucleotide sequence for human CD98HC mRNA is NM_002394.6, and the amino acid sequence for human CD98HC is NP_002385.3 (SEQ ID NO: 2). The location for each exon and each region in human CD98HC nucleotide sequence and amino acid sequence is listed below:

TABLE 1

| | | NP_002385.3 |
|---|---|---|
| Human CD98HC | NM_002394.6 | 630 aa |
| (approximate location) | 2221 bp | (SEQ ID NO: 2) |
| Exon 1 | 1-273 | 1-37 |
| Exon 2 | 274-366 | 38-68 |
| Exon 3 | 367-459 | 69-99 |
| Exon 4 | 460-888 | 100-242 |
| Exon 5 | 889-1062 | 243-300 |
| Exon 6 | 1063-1154 | 301-331 |
| Exon 7 | 1155-1223 | 332-354 |
| Exon 8 | 1224-1282 | 355-374 |
| Exon 9 | 1283-1463 | 375-434 |
| Exon 10 | 1464-1607 | 435-482 |
| Exon 11 | 1608-1691 | 483-510 |
| Exon 12 | 1692-2221 | 511-630 |
| Extracellular | 777-2051 | 206-630 |
| Transmembrane | 714-776 | 185-205 |
| Cytoplasmic | 162-713 | 1-184 |
| Donor region in Example | 804-2054 | 215-630 |

The human CD98HC gene (Gene ID: 6520) is located in Chromosome 11 of the human genome, which is located from 62,856,109 to 62888860 (GRCh38.p13 (GCF_000001405.39)). The 5′-UTR is from 62,856,102 to 62856269, exon 1 is from 62,856,102 to 62,856,381, exon 2 is from 62,870,745 to 62,870,837, exon 3 is from 62,871,577 to 62,871,669, exon 4 is from 62,881,019 to 62,881,447, exon 5 is from 62,881,893 to 62,882,066, exon 6 is from 62,882,908 to 62,882,999, exon 7 is from 62,884,457 to 62,884,525, exon 8 is from 62,884,632 to 62,884,690, exon 9 is from 62,885,177 to 62,885,357, exon 10 is from 62,885,465 to 62,885,608, exon 11 is from 62,888,135 to 62,888,218, exon 12 is from 62,888,331 to 62,888,793, The 3′-UTR is from 62/888,694 to 62,888,793, based on transcript NM_002394.6. All relevant information for human CD98HC locus can be found in the NCBI website with Gene ID: 6520, which is incorporated by reference herein in its entirety.

In mice, CD98HC gene locus has 10 exons, exon 1, exon 2, exon 3, exon 4, exon 5, exon 6, exon 7, exon 8, exon 9, and exon 10 (FIG. 1). The mouse CD98HC protein also has, from N-terminus to C-terminus, an extracellular region, a transmembrane region, and a cytoplasmic region. The nucleotide sequence for mouse CD98HC mRNA is NM_001161413.1, the amino acid sequence for mouse CD98HC is NP_001154885.1 (SEQ ID NO: 1). The location for each exon and each region in the mouse CD98HC nucleotide sequence and amino acid sequence is listed below:

TABLE 2

| | | NP_001154885.1 |
|---|---|---|
| Mouse CD98HC | NM_001161413.1 | 565aa |
| (approximate location) | 2813bp | (SEQ ID NO: 1) |
| Exon 1 | 1-386 | 1-37 |
| Exon 2 | 387-797 | 38-174 |
| Exon 3 | 798-971 | 175-232 |
| Exon 4 | 972-1066 | 233-264 |
| Exon 5 | 1067-1135 | 265-287 |
| Exon 6 | 1136-1194 | 288-307 |
| Exon 7 | 1195-1375 | 308-367 |
| Exon 8 | 1376-1516 | 368-414 |
| Exon 9 | 1517-1600 | 415-442 |
| Exon 10 | 1601-2813 | 443-565 |
| Extracellular | 689-1969 | 139-565 |
| Transmembrane | 617-688 | 115-138 |
| Cytoplasmic | 275-616 | 1-114 |
| Replaced region in Example | 713-1972 | 147-565 |

The mouse CD98HC gene (Gene ID: 17254) is located in Chromosome 19 of the mouse genome, which is located from 8684246 to 8700733 (GRCm39 (GCF_000001635.27)). The 5′-UTR is from 8,700,733 to 8700460, exon 1 is from 8,700,733 to 8,700,348, exon 2 is from 8,691,116 to 8,690,706, exon 3 is from 8,690,267 to 8,690,094, exon 4 is from 8,689,292 to 8,689,198, exon 5 is from 8,687,147 to 8,687,079, exon 6 is from 8,686,972 to 8,686,914, exon 7 is from 8,686,774 to 8,686,594, exon 8 is from 8,686,509 to 8,686,369, exon 9 is from 8,685,641 to 8,685,558, exon 10 is from 8,685,458 to 8,684,246, The 3′-UTR is from 8685086 to 8,684,246, based on transcript NM_001161413.1. All relevant information for mouse CD98HC locus can be found in the NCBI website with Gene ID: 17254, which is incorporated by reference herein in its entirety.

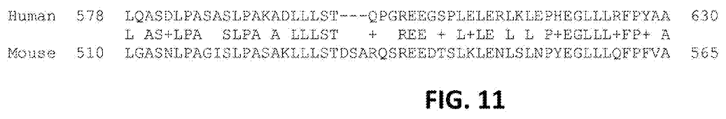

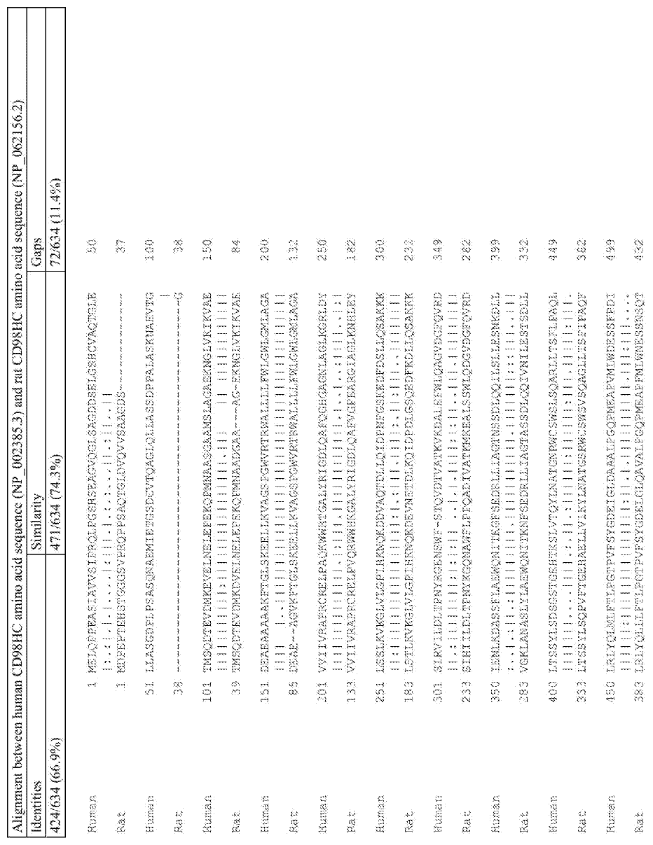

FIG. 11 shows the alignment between human CD98HC amino acid sequence (NP_002385.3; SEQ ID NO: 2) and mouse CD98HC amino acid sequence (NP_001154885.1; SEQ ID NO: 1). Thus, the corresponding amino acid residue or region between human and mouse CD98HC can be found in FIG. 11.

CD98HC genes, proteins, and locus of the other species are also known in the art. For example, the gene ID for CD98HC in Rattus norvegicus (rat) is 50567, the gene ID for CD98HC in Macaca mulatta (Rhesus monkey) is 722218, the gene ID for CD98HC in Canis lupus familiaris (dog) is 483777, and the gene ID for CD98HC in Sus scrofa (pig) is 100144589. The relevant information for these genes (e.g., intron sequences, exon sequences, amino acid residues of these proteins) can be found, e.g., in NCBI database, which is incorporated by reference herein in its entirety.

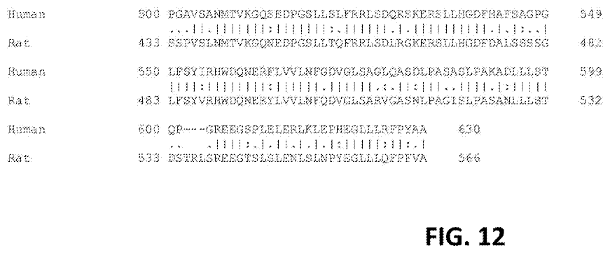

FIG. 12 shows the alignment between human CD98HC amino acid sequence (NP_002385.3; SEQ ID NO: 2) and rat CD98HC amino acid sequence (NP_062156.2; SEQ ID NO: 29). Thus, the corresponding amino acid residue or region between human and rodent CD98HC can be found in FIG. 12.

The present disclosure provides human or chimeric (e.g., humanized) CD98HC nucleotide sequence and/or amino acid sequences. In some embodiments, the entire sequence of mouse exon 1, exon 2, exon 3, exon 4, exon 5, exon 6, exon 7, exon 8, exon 9, exon 10, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, exon 9, exon 10, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 610, 620, 630, 640, 650, 700, 750, 800, 850, 900, 950, 1000, 1050, 1100, 1150, 1200, 1250, or 1260 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 201, 202, 203, 204, 205, 208, 210, 220, 230, 240, 250, 260, 270, 280, 290, 300, 310, 320, 330, 340, 350, 360, 370, 380, 390, 400, 410, or 419 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, extracellular region, transmembrane region, or cytoplasmic region. In some embodiments, a region, a portion, or the entire sequence of mouse exon 1, exon 2, exon 3, exon 4, exon 5, exon 6, exon 7, exon 8, exon 9, and/or exon 10, (e.g., a portion of exon 2, exons 3-9, and a portion of exon 10) are replaced by a region, a portion, or the entire sequence of the human exon 1, exon 2, exon 3, exon 4, exon 5, exon 6, exon 7, exon 8, exon 9, exon 10, exon 11, and/or exon 12 (e.g., a portion of exon 4, exons 5-11, and a portion of exon 12).

In some embodiments, a “region” or “portion” of the extracellular region, transmembrane region, cytoplasmic region, exon 1, exon 2, exon 3, exon 4, exon 5, exon 6, exon 7, exon 8, exon 9, and/or exon 10 is deleted.

In some embodiments, the present disclosure is related to a genetically-modified, non-human animal whose genome comprises a chimeric (e.g., humanized) CD98HC nucleotide sequence. In some embodiments, the chimeric (e.g., humanized) CD98HC nucleotide sequence encodes a CD98HC protein comprising an extracellular region, a transmembrane region, and a cytoplasmic region. In some embodiments, the extracellular region comprises a sequence that is at least 80%, 85%, 90%, 95%, or 100% identical to amino acids 215-630 of SEQ ID NO: 2. In some embodiments, the extracellular region comprises all or part of human CD98HC extracellular region. In some embodiments, the transmembrane region comprises a sequence that is at least 80%, 85%, 90%, 95%, or 100% identical to amino acids 115-138 of SEQ ID NO: 1. In some embodiments, the transmembrane region comprises all or part of endogenous CD98HC transmembrane region. In some embodiments, the cytoplasmic region comprises a sequence that is at least 80%, 85%, 90%, 95%, or 100% identical to amino acids 1-114 of SEQ ID NO: 1. In some embodiments, the cytoplasmic region comprises all or part of endogenous CD98HC cytoplasmic region. In some embodiments, the genome of the animal comprises a sequence that is at least 80%, 85%, 90%, 95%, or 100% identical to SEQ ID NO: 3, 4, 5, 6, 7, 8, 9, 10, 12, or 13.

In some embodiments, the genetically-modified non-human animal described herein comprises a sequence encoding a human or humanized CD98HC protein. In some embodiments, the CD98HC protein comprises, from N-terminus to C-terminus, an extracellular region, a transmembrane region, and a cytoplasmic region. In some embodiments, the humanized CD98HC protein comprises a human or humanized extracellular region. In some embodiments, the humanized CD98HC protein comprises an endogenous extracellular region. In some embodiments, the humanized CD98HC protein comprises a human or humanized transmembrane region. In some embodiments, the humanized CD98HC protein comprises an endogenous transmembrane region. In some embodiments, the humanized CD98HC protein comprises a human or humanized cytoplasmic region. In some embodiments, the humanized CD98HC protein comprises an endogenous cytoplasmic region. In some embodiments, the humanized CD98HC protein comprises a human or humanized extracellular region, an endogenous transmembrane region, and an endogenous cytoplasmic region. In some embodiments, the humanized CD98HC protein comprises an endogenous sequence that corresponds to amino acids 1-146 of SEQ ID NO: 1.

In some embodiments, the genetically-modified non-human animal described herein comprises a human or humanized CD98HC gene. In some embodiments, the humanized CD98HC gene comprises 10 exons. In some embodiments, the humanized CD98HC gene comprises endogenous exon 1 and humanized exons 2-10. In some embodiments, the humanized CD98HC gene comprises 9 introns. In some embodiments, the humanized CD98HC gene comprises endogenous intron 1 and humanized introns 2-9. In some embodiments, the humanized CD98HC gene comprises human or humanized 5′ UTR. In some embodiments, the humanized CD98HC gene comprises human or humanized 3′ UTR. In some embodiments, the humanized CD98HC gene comprises endogenous 5′ UTR. In some embodiments, the humanized CD98HC gene comprises endogenous 3′ UTR.

Thus, in some embodiments, the present disclosure also provides a chimeric (e.g., humanized) CD98HC nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse CD98HC mRNA sequence (e.g., NM_001161413.1), mouse CD98HC amino acid sequence (e.g., SEQ ID NO: 1), or a portion thereof (e.g., 5′ UTR, exon 1, a portion of exon 2, a portion of exon 10, and 3′ UTR); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human CD98HC mRNA sequence (e.g., NM_002394.6), human CD98HC amino acid sequence (e.g., SEQ ID NO: 2), or a portion thereof (e.g., a portion of exon 4, exons 5-11, and a portion of exon 12).

In some embodiments, the sequence encoding amino acids 147-565 of mouse CD98HC (SEQ ID NO: 1) is replaced. In some embodiments, the sequence is replaced by a sequence encoding a corresponding region of human CD98HC (e.g., amino acids 215-630 of human CD98HC (SEQ ID NO: 2)).

In some embodiments, the nucleic acids as described herein are operably linked to a promotor or regulatory element, e.g., an endogenous mouse CD98HC promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 500, 600, 700, 800, 900, 1000, 1100, 1200, 1210, 1220, 1230, 1240, 1250, 1260, 1300, 1400, or 1500 nucleotides, e.g., contiguous or non-contiguous nucleotides) that are different from part of or the entire mouse CD98HC nucleotide sequence (e.g., a portion of exon 2, exons 3-9, and a portion of exon 10 of NM_001161413.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 500, 600, 700, 800, 900, 1000, 1100, 1200, 1300, 1400, 1500, 1510, 1520, 1530, 1540, 1550, 1560, 1600, or 1700 nucleotides, e.g., contiguous or non-contiguous nucleotides) that is the same as part of or the entire mouse CD98HC nucleotide sequence (e.g., 5′ UTR, exon 1, a portion of exon 2, a portion of exon 10, and 3′ UTR of NM_001161413.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 500, 600, 700, 800, 900, 910, 920, 930, 940, 950, 960, 970, 980, 990, or 1000 nucleotides, e.g., contiguous or non-contiguous nucleotides) that is different from part of or the entire human CD98HC nucleotide sequence (e.g., 5′ UTR, exons 1-3, a portion of exon 4, a portion of exon 12, and 3′ UTR of NM_002394.6).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 500, 600, 700, 800, 900, 1000, 1100, 1200, 1210, 1220, 1230, 1240, 1250, 1260, 1270, 1300, or 1400 nucleotides, e.g., contiguous or non-contiguous nucleotides) that is the same as part of or the entire human CD98HC nucleotide sequence (e.g., a portion (at least 50 bp) of exon 4, exons 5-11, and a portion (at least 200 bp) of exon 12 of NM_002394.6).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 410, 419, 420, 430, or 440 amino acid residues, e.g., contiguous or non-contiguous amino acid residues) that is different from part of or the entire mouse CD98HC amino acid sequence (e.g., amino acids 147-565 of NP_001154885.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, or 146, 150, or 160 amino acid residues, e.g., contiguous or non-contiguous amino acid residues) that is the same as part of or the entire mouse CD98HC amino acid sequence (e.g., amino acids 1-146 of NP_001154885.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 214, 220, 230, or 240 amino acid residues, e.g., contiguous or non-contiguous amino acid residues) that is different from part of or the entire human CD98HC amino acid sequence (e.g., amino acids 1-214 of NP_002385.3 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 200, 300, 400, 410, 416, 420, 430, or 440 amino acid residues, e.g., contiguous or non-contiguous amino acid residues) that is the same as part of or the entire human CD98HC amino acid sequence (e.g., amino acids 215-630 of NP_002385.3 (SEQ ID NO: 2)).

The present disclosure also provides a humanized CD98HC amino acid sequence, wherein the amino acid sequence is selected from the group consisting of:
a) an amino acid sequence shown in SEQ ID NO: 1, 2, or 11;
b) an amino acid sequence having a homology of at least 90% with or at least 90% identical to the amino acid sequence shown in SEQ ID NO: 1, 2, or 11;
c) an amino acid sequence encoded by a nucleic acid sequence, wherein the nucleic acid sequence is able to hybridize to a nucleotide sequence encoding the amino acid shown in SEQ ID NO: 1, 2, or 11 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1, 2, or 11;
e) an amino acid sequence that is different from the amino acid sequence shown in SEQ ID NO: 1, 2, or 11 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 1, 2, or 11.

The present disclosure also provides a humanized CD98HC amino acid sequence, wherein the amino acid sequence contains:
a) all or part of amino acids 215-630 of SEQ ID NO: 2;
b) an amino acid sequence have a homology of at least 90%, 91%, 92%, 93%, 94%, 95%, 96%, 97%, 98%, or 99% to amino acids 215-630 of SEQ ID NO: 2;
c) an amino acid sequence that is different from amino acids 215-630 of SEQ ID NO: 2 by no more than 10, 9, 8, 7, 6, 5, 4, 3, 2 or no more than 1 amino acid; or
d) an amino acid sequence that comprises a substitution, a deletion and/or insertion of one or more amino acids to amino acids 215-630 of SEQ ID NO: 2.

The present disclosure also provides a humanized CD98HC amino acid sequence, wherein the amino acid sequence contains:
a) all or part of amino acids 1-146 of SEQ ID NO: 1;
b) an amino acid sequence have a homology of at least 90%, 91%, 92%, 93%, 94%, 95%, 96%, 97%, 98%, or 99% to amino acids 1-146 of SEQ ID NO: 1;
c) an amino acid sequence that is different from amino acids 1-146 of SEQ ID NO: 1 by no more than 10, 9, 8, 7, 6, 5, 4, 3, 2 or no more than 1 amino acid; or
d) an amino acid sequence that comprises a substitution, a deletion and/or insertion of one or more amino acids to amino acids 1-146 of SEQ ID NO: 1.

The present disclosure also relates to a CD98HC nucleic acid (e.g., DNA or RNA) sequence, wherein the nucleic acid sequence can be selected from the group consisting of:
a) a nucleic acid sequence as shown in SEQ ID NO: 3, 4, 5, 6, 7, 8, 9, 10, 12, or 13, or a nucleic acid sequence encoding a homologous CD98HC amino acid sequence of a humanized mouse CD98HC;
b) a nucleic acid sequence that is able to hybridize to the nucleotide sequence as shown in SEQ ID NO: 3, 4, 5, 6, 7, 8, 9, 10, 12, or 13 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 7, 8, 9, 10, 12, or 13;
d) a nucleic acid sequence that encodes an amino acid sequence, wherein the amino acid sequence has a homology of at least 90% with or at least 90% identical to the amino acid sequence shown in SEQ ID NO: 1, 2, or 11;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 2, or 11;
f) a nucleic acid sequence that encodes an amino acid sequence, wherein the amino acid sequence is different from the amino acid sequence shown in SEQ ID NO: 1, 2, or 11 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 1, 2, or 11.

The present disclosure further relates to a CD98HC genomic DNA sequence of a humanized mouse. The DNA sequence is obtained by reverse transcription of the mRNA obtained by transcription thereof is consistent with or complementary to the DNA sequence homologous to the sequence shown in SEQ ID NO: 10.

The disclosure also provides an amino acid sequence that has a homology of at least 90% with, or at least 90% identical to the sequence shown in SEQ ID NO: 1, 2, or 11, and has protein activity. In some embodiments, the homology with the sequence shown in SEQ ID NO: 1, 2, or 11 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 2, or 11 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 10, and encodes a polypeptide that has protein activity. In some embodiments, the homology with the sequence shown in SEQ ID NO: 10 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 3, 4, 5, 6, 7, 8, 9, 10, 12, or 13 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10, 1%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) CD98HC from an endogenous non-human CD98HC locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise a modified endogenous CD98HC locus that comprises an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be e.g., a human exon or a chimeric exon.

In some embodiments, the chimeric gene or the chimeric nucleic acid is a humanized CD98HC gene or a humanized CD98HC nucleic acid. In some embodiments, at least one or more portions of the gene or the nucleic acid is from the human CD98HC gene, at least one or more portions of the gene or the nucleic acid is from a non-human CD98HC gene. In some embodiments, the gene or the nucleic acid comprises a sequence that encodes an CD98HC protein. The encoded CD98HC protein is functional or has at least one activity of the human CD98HC protein or the non-human CD98HC protein, e.g., binding to integrins and CD98 light chain.

In some embodiments, the chimeric protein or the chimeric polypeptide is a humanized CD98HC protein or a humanized CD98HC polypeptide. In some embodiments, at least one or more portions of the amino acid sequence of the protein or the polypeptide is from a human CD98HC protein, and at least one or more portions of the amino acid sequence of the protein or the polypeptide is from a non-human CD98HC protein. The humanized CD98HC protein or the humanized CD98HC polypeptide is functional or has at least one activity of the human CD98HC protein or the non-human CD98HC protein.

In some embodiments, the extracellular region is human or humanized. In some embodiments, the cytoplasmic region is human or humanized. In some embodiments, the transmembrane region is human or humanized. In some embodiments, both the transmembrane and cytoplasmic regions are endogenous.

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Ca, CBA/J, CBA/st, or CBA/H background.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized CD98HC animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγ knockout mice, NOD/SCID/γc^{null} mice (Ito, M. et al., NOD/SCID/γc^{null} mouse: an excellent recipient mouse model for engraftment of human cells, Blood 100(9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of an endogenous non-human CD98HC locus, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2Rγ knockout mice, NOD/SCID/γc^{null} mice, nude mice, Rag1 and/or Rag2 knockout mice, NOD-Prkdc^{scid} IL-2rγ^{null} mice, NOD-Rag 1^{-/-}-IL2rg^{-/-} (NRG) mice, Rag 2^{-/-}-IL2rg^{-/-} (RG) mice, and a combination thereof. These genetically modified animals are described, e.g., in US20150106961, which is incorporated herein by reference in its entirety. In some embodiments, the mouse can include a replacement of all or part of CD98HC coding sequence with human CD98HC coding sequence.

Genetically modified non-human animals can comprise a modification at an endogenous non-human CD98HC locus. In some embodiments, the modification can comprise a human nucleic acid sequence encoding at least a portion of a mature CD98HC protein (e.g., at least 10%, 20%, 30%, 40%, 50%, 60%, 70%, 80%, 90%, 95%, 96%, 97%, 98%, or 99% identical to the CD98HC protein sequence). Although genetically modified cells are also provided that can comprise the modifications described herein (e.g., ES cells, somatic cells), in many embodiments, the genetically modified non-human animals comprise the modification of the endogenous CD98HC locus in the germline of the animal.

Genetically modified animals can express a human CD98HC and/or a chimeric (e.g., humanized) CD98HC from endogenous mouse loci, wherein the endogenous mouse CD98HC gene has been replaced with a human CD98HC gene and/or a nucleotide sequence that encodes a region of human CD98HC sequence or an amino acid sequence that is at least 10%, 20%, 30%, 40%, 50%, 60%, 70&, 80%, 90%, 95%, 96%, 97%, 98%, or 99% identical to the human CD98HC sequence. In various embodiments, an endogenous non-human CD98HC locus is modified in whole or in part to comprise human nucleic acid sequence encoding at least one protein-coding sequence of a mature CD98HC protein.

In some embodiments, the genetically modified mice can express the human CD98HC and/or chimeric CD98HC (e.g., humanized CD98HC) from endogenous loci that are under control of mouse promoters and/or mouse regulatory elements. The replacement(s) at the endogenous mouse loci provide non-human animals that express human CD98HC or chimeric CD98HC (e.g., humanized CD98HC) in appropriate cell types and in a manner that does not result in the potential pathologies observed in some other transgenic mice known in the art. The human CD98HC or the chimeric CD98HC (e.g., humanized CD98HC) expressed in animal can maintain one or more functions of the wild-type mouse or human CD98HC in the animal. For example, the expressed CD98HC can bind to human integrins or non-human (e.g., mouse) integrins. In some embodiments, the expressed CD98HC can bind to human CD98 light chain or non-human (e.g., mouse) CD98 light chain. Furthermore, in some embodiments, the animal does not express endogenous CD98HC. In some embodiments, the animal expresses a decreased level of endogenous CD98HC as compared to CD98HC expression level in a wild-type animal. As used herein, the term “endogenous CD98HC” refers to CD98HC protein that is expressed from an endogenous CD98HC nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human CD98HC (NP_002385.3) (SEQ ID NO: 2). In some embodiments, the genome comprises a sequence encoding an amino acid sequence that is at least 70%, 75%, 80%, 85%, 90%, 95%, 99%, or 100% identical to SEQ ID NO: 11.

The genome of the genetically modified animal can comprise a replacement at an endogenous CD98HC gene locus of a sequence encoding a region of endogenous CD98HC with a sequence encoding a corresponding region of human CD98HC. In some embodiments, the sequence that is replaced is any sequence within the endogenous CD98HC gene locus, e.g., exon 1, exon 2, exon 3, exon 4, exon 5, exon 6, exon 7, exon 8, exon 9, exon 10, 5′-UTR, 3′-UTR, intron 1, intron 2, intron 3, intron 4, intron 5, intron 6, intron 7, intron 8, intron 9, or any combination thereof. In some embodiments, the sequence that is replaced is within the regulatory region of the endogenous CD98HC gene. In some embodiments, the sequence that is replaced is a portion of exon 2, exons 3-9, and a portion of exon 10, of an endogenous mouse CD98HC gene locus.

The genetically modified animal can have one or more cells expressing a human or chimeric CD98HC (e.g., humanized CD98HC) having, from N-terminus to C-terminus, an extracellular region, a transmembrane region, and a cytoplasmic region. In some embodiments, the extracellular region comprises a sequence that is at least 50%, 60%, 70%, 80%, 90%, 95%, 99% identical to the extracellular region of human CD98HC. In some embodiments, the extracellular region of the humanized CD98HC has a sequence that has at least 10, 20, 30, 40, 50, 60, 70, 80, 90, 100, 110, 120, 130, 140, 150, 160, 170, 180, 190, 200, 250, 300, 350, 400, 410, or 416 amino acids (e.g., contiguously or non-contiguously) that are identical to the extracellular region of human CD98HC. Because human CD98HC and non-human CD98HC (e.g., mouse CD98HC) sequences, in many cases, are different, antibodies that bind to human CD98HC will not necessarily have the same binding affinity with non-human CD98HC or have the same effects to non-human CD98HC. Therefore, the genetically modified animal having a human or a humanized extracellular region can be used to better evaluate the effects of anti-human CD98HC antibodies in an animal model.

In some embodiments, the transmembrane comprises a sequence that is at least 50%, 60%, 70%, 80%, 90%, 95%, 99% identical to the transmembrane region of endogenous CD98HC (e.g., amino acids 115-138 of SEQ ID NO: 1). In some embodiments, the transmembrane region of the humanized CD98HC has a sequence that has at least 1, 2, 3, 4, 5, 6, 7, 8, 9, 10, 11, 12, 13, 14, 15, 16, 17, 18, 19, 20, 21, 22, 23, or 24 amino acids (contiguously or non-contiguously) that are identical to the transmembrane region of endogenous CD98HC (e.g., mouse CD98HC). In some embodiments, the cytoplasmic comprises a sequence that is at least 50%, 60%, 70%, 80%, 90%, 95%, 99% identical to the cytoplasmic of endogenous CD98HC (e.g., amino acids 1-114 of SEQ ID NO: 1). In some embodiments, the cytoplasmic region of the humanized CD98HC has a sequence that has at least 10, 20, 30, 40, 50, 60, 70, 80, 90, 100, 110, 111, 112, 113, or 114 amino acids (contiguously or non-contiguously) that are identical to the cytoplasmic region of endogenous CD98HC (e.g., mouse CD98HC).

In some embodiments, the entire transmembrane region and the entire cytoplasmic region of the humanized CD98HC described herein are derived from endogenous sequence.

In some embodiments, the genome of the genetically modified animal comprises a sequence encoding an amino acid sequence that corresponds to a portion or the entire sequence of exon 1, exon 2, exon 3, exon 4, exon 5, exon 6, exon 7, exon 8, exon 9, exon 10, exon 11, and/or exon 12 of human CD98HC; a portion or the entire sequence of the extracellular region of human CD98HC; or a portion or the entire sequence of amino acids 215-630 of SEQ ID NO: 2.

In some embodiments, the genome of the genetically modified animal comprises a portion of exon 4, exons 5-11, and a portion of exon 12 of human CD98HC gene. In some embodiments, the portion of exon 4 includes at least 5, 10, 15, 20, 25, 30, 35, 40, 45, 46, 47, 48, 49, 50, 55, 60, 61, 62, 63, 64, 65, 70, 80, 81, 82, 83, 84 or 85 nucleotides. In some embodiments, the portion of exon 4 includes 85 nucleotides. In some embodiments, the portion of exon 4 includes a nucleotide of at least 50 bp. In some embodiments, the portion of exon 12 includes at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 200, 250, 300, 310, 320, 330, 340,350, 360, or 363 nucleotides. In some embodiments, the portion of exon 12 includes 363 nucleotides. In some embodiments, the portion of exon 12 includes a nucleotide of at least 200 bp.

In some embodiments, the non-human animal can have, at an endogenous CD98HC gene locus, a nucleotide sequence encoding a chimeric human/non-human CD98HC polypeptide, wherein a human portion of the chimeric human/non-human CD98HC polypeptide comprises all or a portion of the human CD98HC extracellular region, and wherein the animal expresses a functional CD98HC on a surface of a cell of the animal. The human portion of the chimeric human/non-human CD98HC polypeptide can comprise an amino acid sequence encoded by a portion of exon 4, exons 5-11, and/or a portion of exon 12 of human CD98HC. In some embodiments, the human portion of the chimeric human/non-human CD98HC polypeptide can comprise a sequence that is at least 80%, 85%, 90%, 95%, or 99% identical to amino acids 215-630 of SEQ ID NO: 2. In some embodiments, the transmembrane region includes a sequence corresponding to the entire or part of amino acids 115-138 of SEQ ID NO: 1. In some embodiments, the cytoplasmic region includes a sequence corresponding to the entire or part of amino acids 1-114 of SEQ ID NO: 1.

In some embodiments, the non-human portion of the chimeric human/non-human CD98HC polypeptide comprises the entire transmembrane region and/or the entire cytoplasmic region of an endogenous non-human CD98HC polypeptide.

Furthermore, the genetically modified animal can be heterozygous with respect to the replacement at the endogenous CD98HC locus, or homozygous with respect to the replacement at the endogenous CD98HC locus.

In some embodiments, the humanized CD98HC locus lacks a human CD98HC 5′-UTR. In some embodiment, the humanized CD98HC locus comprises an endogenous (e.g., mouse) 5′-UTR. In some embodiments, the humanization comprises an endogenous (e.g., mouse) 3′-UTR. In appropriate cases, it may be reasonable to presume that the mouse and human CD98HC genes appear to be similarly regulated based on the similarity of their 5′-flanking sequence. As shown in the present disclosure, humanized CD98HC mice that comprise a replacement at an endogenous mouse CD98HC locus, which retain mouse regulatory elements but comprise a humanization of CD98HC encoding sequence, do not exhibit pathologies. Both genetically modified mice that are heterozygous or homozygous for humanized CD98HC are grossly normal.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by a humanized CD98HC gene.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized CD98HC in the genome of the animal.

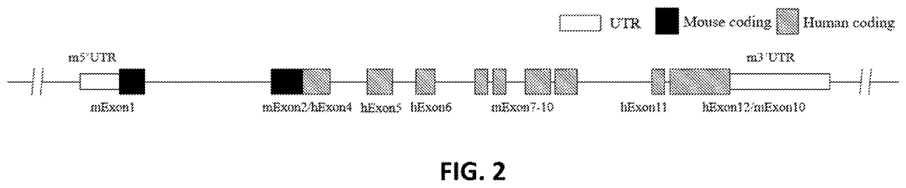

In some embodiments, the non-human mammal comprises the genetic construct as described herein (e.g., gene construct as shown in FIGS. 2, 3, and 4). In some embodiments, a non-human mammal expressing human or humanized CD98HC is provided. In some embodiments, the tissue-specific expression of human or humanized CD98HC protein is provided.

In some embodiments, the expression of human or humanized CD98HC in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human CD98HC protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized CD98HC protein.

In another aspect, the disclosure also provides a genetically-modified, non-human animal whose genome comprise a disruption in the animal's endogenous CD98HC gene, wherein the disruption of the endogenous CD98HC gene comprises deletion of exon 1, exon 2, exon 3, exon 4, exon 5, exon 6, exon 7, exon 8, exon 9, and/or exon 10, or part thereof of the endogenous CD98HC gene.

In some embodiments, the disruption of the endogenous CD98HC gene comprises deletion of one or more exons or part of exons selected from the group consisting of exon 1, exon 2, exon 3, exon 4, exon 5, exon 6, exon 7, exon 8, exon 9, and exon 10 of the endogenous CD98HC gene.

In some embodiments, the disruption of the endogenous CD98HC gene further comprises deletion of one or more introns or part of introns selected from the group consisting of intron 1, intron 2, intron 3, intron 4, intron 5, intron 6, intron 7, intron 8, and intron 9 of the endogenous CD98HC gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 5000, 6000, 6500, or more nucleotides.

In some embodiments, the disruption of the endogenous CD98HC gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 400, 500, 600, 700, 800, 900, 1000, 1100, 1200, 1250, or 1260 nucleotides of exon 1, exon 2, exon 3, exon 4, exon 5, exon 6, exon 7, exon 8, exon 9, and/or exon 10, (e.g., deletion of at least 20 nucleotides from exon 2, exons 3-9, and at least 100 nucleotides from exon 10).

### Vectors

The present disclosure relates to a targeting vector, comprising: a) a DNA fragment homologous to the 5′ end of a region to be altered (5′ arm), which is selected from the CD98HC gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the CD98HC gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000085.7; c) the DNA fragment homologous to the 3′ end of the region to be altered (3′ arm) is selected from the nucleotide sequences that have at least 90% homology to the NCBI accession number NC_000085.7.

In some embodiments, a) the DNA fragment homologous to the 5′ end of a region to be altered (5′ arm) is selected from the nucleotides from the position 8690791 to the position 8694207 of the NCBI accession number NC_000085.7; c) the DNA fragment homologous to the 3′ end of the region to be altered (3′ arm) is selected from the nucleotides from the position 8678908 to the position 8683721 of the NCBI accession number NC_000085.7.

In some embodiments, a) the DNA fragment homologous to the 5′ end of a region to be altered (5′ arm) is selected from the nucleotides from the position 8690791 to the position 8691758 of the NCBI accession number NC_000085.7; c) the DNA fragment homologous to the 3′ end of the region to be altered (3′ arm) is selected from the nucleotides from the position 8683723 to the position 8685086 of the NCBI accession number NC_000085.7.

In some embodiments, the length of the selected genomic nucleotide sequence in the targeting vector can be more than about 3 kb or more, more than about 4 kb, more than about 5 kb, more than about 6 kb or more than about 7 kb.

In some embodiments, the region to be altered is exon 1, exon 2, exon 3, exon 4, exon 5, exon 6, exon 7, exon 8, exon 9, and/or exon 10 of CD98HC gene (e.g., a portion of exon 2, exons 3-9, and a portion of exon 10 of mouse CD98HC gene).

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the sequence of the 5′ arm is shown in SEQ ID NO: 3; and the sequence of the 3′ arm is shown in SEQ ID NO: 4. In some embodiments, the sequence of the 5′ arm is shown in SEQ ID NO: 5; and the sequence of the 3′ arm is shown in SEQ ID NO: 6.

In some embodiments, the donor sequence is derived from human (e.g., 62881363-62888693 of NC_000011.10; or 804-2054 of NM_002394.6). For example, the target region in the targeting vector is a part or entirety of the nucleotide sequence of a human CD98HC, preferably exon 1, exon 2, exon 3, exon 4, exon 5, exon 6, exon 7, exon 8, exon 9, exon 10, exon 11, and/or exon 12 of the human CD98HC. In some embodiments, the nucleotide sequence of the humanized CD98HC encodes the entire or the part of human CD98HC protein with the NCBI accession number NP_002385.3 (SEQ ID NO: 2).

The disclosure also provides vectors for constructing a humanized animal model or a knock-out model. In some embodiments, the vectors comprise a sgRNA sequence, wherein the sgRNA sequence targets CD98HC gene, and the sgRNA is unique on the target sequence of the gene to be altered, and meets the sequence arrangement rule of 5′-NNN (20)-NGG3′ or 5′-CCN-N (20)-3′; and in some embodiments, the targeting site of the sgRNA in the mouse CD98HC gene is located on the exon 1, exon 2, exon 3, exon 4, exon 5, exon 6, exon 7, exon 8, exon 9, exon 10, intron 1, intron 2, intron 3, intron 4, intron 5, intron 6, intron 7, intron 8, intron 9, upstream of exon 1, or downstream of exon 10 of the mouse CD98HC gene.

In some embodiments, the targeting sequences are shown as SEQ ID NOs: 12 and 13. Thus, the disclosure provides sgRNA sequences for constructing a genetic modified animal model.

In some embodiments, the disclosure relates to a plasmid construct (e.g., pT7-sgRNA) including the sgRNA sequence, and/or a cell including the construct.

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous CD98HC gene locus, a sequence encoding a region of an endogenous CD98HC with a sequence encoding a corresponding region of human or chimeric CD98HC. In some embodiments, the replacement occurs in a germ cell, a somatic cell, a blastocyst, or a fibroblast, etc. The nucleus of a somatic cell or the fibroblast can be inserted into an enucleated oocyte.

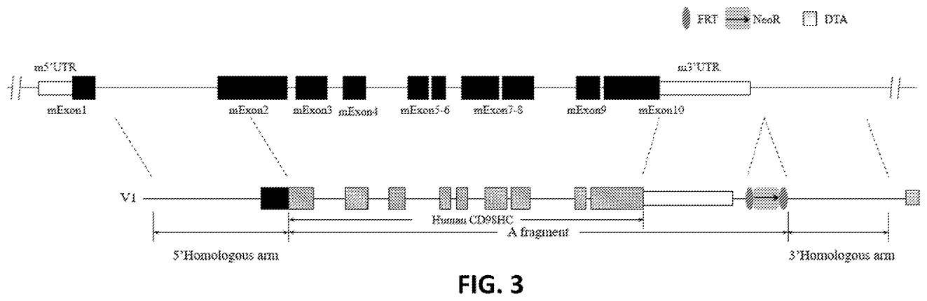

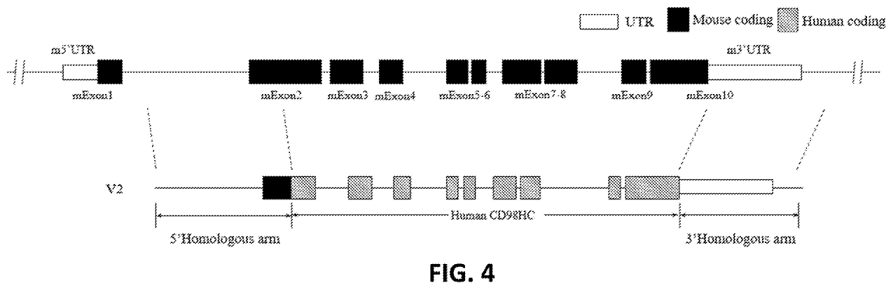

FIG. 3 and FIG. 4 show humanization strategies for a mouse CD98HC locus. The targeting strategies involve a vector comprising a 5′ homologous arm, a human CD98HC gene fragment, and a 3′ homologous arm. The process can involve replacing endogenous CD98HC sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous CD98HC sequence with human CD98HC sequence.

Thus, in some embodiments, the methods for making a genetically modified, humanized animal, can include the step of replacing at an endogenous CD98HC locus (or site), a nucleic acid sequence encoding a region of endogenous CD98HC with a sequence encoding a corresponding region of human CD98HC. The sequence can include a region (e.g., a part or the entire region) of exon 1, exon 2, exon 3, exon 4, exon 5, exon 6, exon 7, exon 8, exon 9, exon 10, exon 11, and/or exon 12 of a human CD98HC gene. In some embodiments, the sequence includes a portion of exon 4, exons 5-11, and a portion of exon 12 of a human CD98HC gene (e.g., nucleic acids 804-2054 of NM_002394.6). In some embodiments, the region includes the extracellular region of human CD98HC (e.g., amino acids 215-630 of SEQ ID NO: 2). In some embodiments, the endogenous CD98HC locus is exon 1, exon 2, exon 3, exon 4, exon 5, exon 6, exon 7, exon 8, exon 9, and/or exon 10, of mouse CD98HC. In some embodiments, the nucleic acid sequence encoding a region of endogenous CD98HC includes a portion of exon 2, exons 3-9, and a portion of exon 10 of mouse CD98HC gene (e.g., nucleic acids 713-1972 of NM_001161413.1).

In some embodiments, the methods of modifying a CD98HC locus of a mouse to express a chimeric human/mouse CD98HC peptide can include the steps of replacing at the endogenous mouse CD98HC locus a nucleotide sequence encoding a mouse CD98HC with a nucleotide sequence encoding a human CD98HC, thereby generating a sequence encoding a chimeric human/mouse CD98HC.

In some embodiments, the nucleotide sequence encoding the chimeric human/mouse CD98HC can include a first nucleotide sequence encoding all or a portion of the extracellular region of human CD98HC; and a second nucleotide sequence encoding the transmembrane region and the cytoplasmic region of mouse CD98HC.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a CD98HC gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's CD98HC gene, e.g., by inserting a nucleotide sequence (e.g., DNA or cDNA sequence) encoding human or humanized CD98HC protein, e.g., immediately after the endogenous regulatory element of the non-human animal's CD98HC gene. For example, one or more functional region sequences of the non-human animal's CD98HC gene can be knocked out, or inserted with a sequence, such that the non-human animal cannot express or expresses a decreased level of endogenous CD98HC protein. In some embodiments, the coding frame of the modified non-human animal's CD98HC gene can be all or part of the nucleotide sequence from exon 1 to exon 10 of the non-human animal's CD98HC gene.

In some embodiments, methods of making the genetically modified animal comprises inserting a nucleotide sequence encoding human or humanized CD98HC protein and/or an auxiliary sequence after the endogenous regulatory element of the non-human animal's CD98HC gene. In some embodiments, the auxiliary sequence can be a stop codon, such that the CD98HC gene humanized animal model can express human or humanized CD98HC protein in vivo, but does not express non-human animal's CD98HC protein. In some embodiments, the auxiliary sequence includes WPRE (WHP Posttranscriptional Response Element), loxP, and/or polyA.

In some embodiments, the method for making the genetically modified animal comprises:
(1) providing a plasmid comprising a human CD98HC gene fragment, flanked by a 5′ homologous arm and a 3′ homologous arm, wherein the 5′ and 3′ homologous arms target an endogenous CD98HC gene;
(2) providing one or more small guide RNAs (sgRNAs) that target the endogenous CD98HC gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized CD98HC protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized CD98HC protein is operably linked to an endogenous regulatory element at the endogenous CD98HC gene locus.

In some embodiments, the genetically-modified animal does not express an endogenous CD98HC protein.

In some embodiments, the method for making the genetically modified animal comprises:
(1) providing a plasmid comprising a human or chimeric CD98HC gene fragment, flanked by a 5′ homologous arm and a 3′ homologous arm, wherein the 5′ and 3′ homologous arms target an endogenous CD98HC gene;
(2) providing one or more small guide RNAs (sgRNAs) that target the endogenous CD98HC gene; and
(3) modifying genome of a fertilized egg or an embryonic stem cell by inserting the human or chimeric CD98HC gene fragment into the genome.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized CD98HC protein, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized CD98HC, which are useful for testing therapeutic agents that can decrease or block the interaction between the interaction between CD98HC and anti-human CD98HC antibodies, testing whether an therapeutic agent can increase or decrease the immune response, and/or determining whether an agent is an CD98HC agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor (e.g., breast cancer) or a blood cell tumor (e.g., a lymphocyte tumor, a B or T cell tumor).

In some embodiments, the genetically modified animals can be used for determining effectiveness of a therapeutic agent (e.g., an anti-CD98HC antibody or a CD98HC-targeting drug) for the treatment of cancer. In some embodiments, the methods involve administering the therapeutic agent (e.g., an anti-human CD98HC antibody or a CD98HC-targeting drug) to the animal as described herein, wherein the animal has a cancer or tumor; and determining inhibitory effects of the therapeutic agent to the cancer or tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In addition, a delicate balance is required for these antibodies, as CD98HC is also expressed on many other cells. Thus, it is important that the humanized CD98HC functions in a largely similar way as compared to the endogenous CD98HC, so that the results in the humanized animals can be used to predict the efficacy or toxicity of these therapeutic agents in the human. In some embodiments, the anti-CD98HC antibody can directly target cancer cells or tumor-associated cells expressing CD98HC, e.g., by inducing complement mediated cytotoxicity (CMC) or antibody dependent cellular cytotoxicity (ADCC) to kill the cancer cells.

In some embodiments, the tumor comprises one or more cancer cells (e.g., human or mouse cancer cells) that are injected into the animal. In some embodiments, the anti-CD98HC antibody inhibits CD98HC signaling pathways. In some embodiments, the anti-CD98HC antibody does not inhibit CD98HC signaling pathways. In some embodiments, the anti-CD98HC antibody activates CD98HC signaling pathways.

In some embodiments, the genetically modified animals can be used for determining whether an anti-CD98HC antibody is a CD98HC agonist or antagonist. In some embodiments, the methods as described herein are also designed to determine the effects of the therapeutic agent (e.g., anti-CD98HC antibodies) on CD98HC, e.g., whether the agent can block the interaction of CD98HC and integrins, whether the agent can deplete CD98HC-expressing cells (e.g., T cells), whether the agent can induce apoptosis of CD98HC-expressing immune cells, whether the agent can upregulate the immune response or downregulate immune response, and/or whether the agent can induce complement mediated cytotoxicity (CMC) or antibody dependent cellular cytotoxicity (ADCC). In some embodiments, the genetically modified animals can be used for determining the effective dosage of a therapeutic agent for treating a disease in the subject, e.g., an autoimmune disease or cancer.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the therapeutic agent (e.g., an anti-CD98HC antibody or a CD98HC-targeting drug) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In a specific embodiment, the tumor is breast cancer, ovarian cancer, endometrial cancer, melanoma, kidney cancer, lung cancer, or liver cancer.

In some embodiments, the cancer described herein is glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, or melanoma. In some embodiments, the cancer described herein is lymphoma, bladder cancer, breast cancer, lung cancer, squamous cell carcinoma, intestinal adenoma, or leukemia.

In some embodiments, the therapeutic agent (e.g., an anti-CD98HC antibody or a CD98HC-targeting drug) is designed for treating various autoimmune diseases, including rheumatoid arthritis, Crohn's disease, systemic lupus erythematosus, ankylosing spondylitis, inflammatory bowel diseases (IBD), ulcerative colitis, or scleroderma. In some embodiments, the anti-CD98HC antibody is designed for treating various immune disorders, including allergy, asthma, and/or atopic dermatitis. Thus, the methods as described herein can be used to determine the effectiveness of an therapeutic agent (e.g., an anti-CD98HC antibody or a CD98HC-targeting drug) in inhibiting immune response. In some embodiments, the immune disorders described herein is graft versus host disease (GVHD), psoriasis, allergy, asthma, myocarditis, nephritis, hepatitis, systemic lupus erythematosus, rheumatoid arthritis, scleroderma, hyperthyroidism, idiopathic thrombocytopenic purpura, autoimmune hemolytic anemia, ulcerative colitis, autoimmune liver disease, diabetes, pain or neurological disorders, etc. In some embodiments, the methods as described herein can be used to determine the effectiveness of an therapeutic agent (e.g., an anti-CD98HC antibody or a CD98HC-targeting drug) in preventing and/or ameliorating immune recognition and attack to transplanted organs.

The present disclosure also provides methods of determining toxicity of an antibody (e.g., anti-CD98HC antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the CD98HC gene function, human CD98HC antibodies, drugs for human CD98HC targeting sites, the drugs or efficacies for human CD98HC targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the CD98HC gene humanized non-human animal prepared by the methods described herein, the CD98HC gene humanized non-human animal described herein, the double- or multi-humanized non-human animal generated by the methods described herein (or progeny thereof), a non-human animal expressing the human or humanized CD98HC protein, or the tumor-bearing or inflammatory animal models described herein. In some embodiments, the TCR-T, CAR-T, and/or other immunotherapies can treat the CD98HC-associated diseases described herein. In some embodiments, the TCA-T, CAR-T, and/or other immunotherapies provides an evaluation method for treating the CD98HC-associated diseases described herein.

Genetically Modified Animal Model with Two or More Human or Chimeric Genes

The present disclosure further relates to methods for generating genetically modified animal model with two or more human or chimeric genes. The animal can comprise a human or chimeric CD98HC gene and a sequence encoding an additional human or chimeric protein.

In some embodiments, the additional human or chimeric protein can be programmed cell death protein 1 (PD-1), programmed cell death ligand 1 (PD-L1), lymphocyte-activation gene 3 (LAG3), tumor necrosis factor receptor superfamily member 9 (4-1BB), CD40, cytotoxic T-lymphocyte-associated protein 4 (CTLA4), CD147, Interleukin 6 receptor (IL6R), Interleukin 17 (IL17), CD3, CD28, or CD38.

The methods of generating genetically modified animal model with two or more human or chimeric genes (e.g., humanized genes) can include the following steps:
(a) using the methods of introducing human CD98HC gene or chimeric CD98HC gene as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL4, IL4R, OX40, TIGIT, CD27, CD73, PD-1, PD-L1, LAG3, 4-1BB, CD40, CTLA4, CD147, IL6, IL6R, IL17, CD3, CD28 or CD38. Some of these genetically modified non-human animal are described, e.g., in PCT/CN2018/120713, PCT/CN2018/091846, PCT/CN2019/110819, PCT/CN/2019/126045, PCT/CN2017/090320, PCT/CN2017/099577, PCT/CN2017/099575, PCT/CN2017/099576, PCT/CN2017/099574, PCT/CN2017/110435, PCT/CN2017/120388, PCT/CN2017/117984, PCT/CN2018/091845, and PCT/CN2019/119793; each of which is incorporated herein by reference in its entirety.

In some embodiments, the CD98HC humanization is directly performed on a genetically modified animal having a human or chimeric PD-1, PD-L1, LAG3, 4-1BB, CD40, CTLA4, CD147, IL6R, IL17, CD3, CD28, and/or CD38.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-CD98HC antibody and an additional therapeutic agent for the treatment of cancer. The methods include administering the anti-CD98HC antibody and the additional therapeutic agent to the animal, wherein the animal has a tumor; and determining the inhibitory effects of the combined treatment to the tumor. In some embodiments, the additional therapeutic agent is an antibody that specifically binds to PD-1, PD-L1, LAG3, 4-1BB, CD40, CTLA4, CD147, IL6R, IL17, CD3, CD28, and/or CD38. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancers as described herein, e.g., a solid tumor, gynecologic cancer, breast cancer, colorectal cancer, gastric adenocarcinoma, lung adenocarcinoma, pancreatic cancer, or head and neck cancer.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

In each of the following examples, equipment and materials were obtained from the companies indicated below:
BbsI, EcoRI, BamHI, EcoRV, and BclI enzymes were purchased from NEB (Catalog numbers: R0539L, R0101M, R0136M, R0144L, and R0132M);
C57BL/6 mice were purchased from the China Food and Drugs Research Institute National Rodent Experimental Animal Center.

Ambion in vitro transcription kit was purchased from Ambion (Catalog number: AM1354);
Cas9 mRNA is from SIGMA (Catalog number: CAS9MRNA-1EA);
The UCA kit is from Biocytogen (Catalog number: BCG-DX-001);
Brilliant Violet 510™ anti-mouse CD45 Antibody (mCD45) was purchased from Biolegend, (Catalog number: 103138);
PerCP/Cy5.5 anti-mouse TCR β chain Antibody (mTCR β) was purchased from Biolegend (Catalog number: 109228);
Brilliant Violet 605™ anti-mouse CD19 Antibody (mCD19) was purchased from Biolegend (Catalog number: 115540);
APC anti-mouse CD98 (4F2) Antibody (mCD98HC) was purchased from Biolegend (Catalog number: 128211);
FITC anti-human CD98 Antibody (hCD98HC) was purchased from Biolegend (Catalog number: 315603);
APC Rat IgG2a, κ Isotype Ctrl Antibody (mouse isotype control antibody) was purchased from Biolegend (Catalog number: 400512);
FITC Mouse IgG1, κ Isotype Ctrl (FC) Antibody (human isotype control antibody) was purchased from Biolegend (Catalog number: 400110);
Zombie NIR™ Fixable Viability Kit was purchased from Biolegend (Catalog Number: 423106);
Purified anti-mouse CD16/32 was purchased from Biolegend (Catalog Number: 101302);
4F2hc/CD98 (D3F9D) XP® Rabbit mAb was purchased from Cell Signaling Technology (Catalog Number: 47213);
Recombinant Anti-CD31 antibody [EPR17259] was purchased from abcam (Catalog Number: ab182981).

### Example 1: CD98HC Gene Humanized Mice

Mouse CD98HC gene (NCBI Gene ID: 17254, Primary source: MGI: 96955, UniProt ID: P10852-2, located at positions 8684246 to 8700733 of chromosome 19 NC_000085.7, based on transcript NM_001161413.1 and its encoded protein NP_001154885.1 (SEQ ID NO: 1)) and human CD98HC gene (NCBI Gene ID: 6520, Primary source: HGNC: 11026, UniProt ID: P08195, located at positions 62856109 to 62/888,860 of chromosome 11 NC_000011.10, based on transcript NM_002394.6 and its encoded protein NP_002385.3 (SEQ ID NO: 2)) are shown in FIG. 1.

To make CD98HC gene humanized mice, a nucleotide sequence encoding human CD98HC protein was introduced into the mouse endogenous CD98HC gene locus, so that the mouse expresses a human or humanized CD98HC protein. Specifically, using gene editing technologies, under the control of the regulatory elements of the mouse CD98HC gene, a nucleotide sequence encoding the human CD98HC protein was used to replace the corresponding mouse sequence. A schematic diagram of the humanized CD98HC gene locus is shown in FIG. 2.

A schematic diagram of the targeting strategy is shown in FIG. 3. FIG. 3 shows the targeting vector V1, which contains the upstream and downstream homology arm sequences, and the A fragment containing the nucleotide sequence encoding the human CD98HC protein. Among them, the upstream homology arm sequence (5′ homology arm, SEQ ID NO: 3) is the same as the nucleotide sequence at positions 8690791-8694207 of NCBI accession number NC_000085.7, and the downstream homology arm sequence (3′ homology arm, SEQ ID NO: 4) is the same as the nucleotide sequence at positions 8678908-8683721 of NCBI accession number NC_000085.7; the human CD98HC sequence in the A fragment (SEQ ID NO: 7) is the same as the nucleotide sequence at positions 62881363 to 62/888,693 of NCBI accession number NC_000011.10.

The targeting vector also includes a resistance gene for screening positive clones, namely the neomycin phosphotransferase coding sequence (Neo), which is flanked by two site-specific recombination systems (Frt) arranged in the same direction, forming a Neo cassette. The connection between the 5′ end of the Neo cassette and the mouse gene is designed as

(SEQ ID NO: 8)
5′-
GGCAGATCACAAAGGGTTTCATAAGGCAGTCAGAGGTGCA
AAGCTTCCTAGGGATATCGAATTCCGAAGTTCCT
ATTCTC-3′,

wherein the “A” in the sequence “GTGCA” is the last nucleotide of mouse sequence, the first “A” in “AAGCT” is the first nucleotide of the Neo cassette. The connection between the 3′ end of the Neo cassette to the mouse gene was designed as 5′-

(SEQ ID NO: 9)
5-′
GTATAGGAACTTCATCAGTCAGGTACATAATGGTGGATCC
CAGTTTGGTAGGCTCTGC
GGGTCTGTGGGACTATCCTAAA-3′,

where the last “C” in the sequence “GATCC” is the last nucleotide of the Neo cassette and the “C” in the sequence “CAGTT” is the first nucleotide of the mouse sequence. In addition, a gene encoding a negative selection marker (the gene encoding the diphtheria toxin A subunit (DTA)) was included downstream of the 3′ homology arm of the targeting vector. The mRNA sequence of the humanized CD98HC is shown in SEQ ID NO: 10, and the expressed protein sequence is shown in SEQ ID NO: 11.

Targeting vector construction was carried out e.g., by enzyme digestion and ligation. The constructed targeting vector was first preliminarily verified by enzyme digestion, and then sent to a sequencing company for sequencing verification. The correct targeting vector verified by sequencing was transfected into the embryonic stem cells of C57BL/6 mice via electroporation. The obtained cells were screened with the positive clone selection marker gene, and PCR and Southern Blot were used to confirm the presence of exogenous genes. Based on the integration of exogenous genes, the correct positive clone cells were selected. The positive clones (black mice) were introduced into isolated blastocysts (white mice). The chimeric blastocysts obtained were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. Positive F1 generation heterozygous mice can also be mated with Flp tool mice to remove the positive clone screening marker gene, and then cross-mated with each other to obtain CD98HC humanized homozygous mice.

The CRISPR/Cas9 system was also be used for gene editing. The targeting strategy is shown in FIG. 4. The sequence of the upstream homology arm (5′ homology arm, SEQ ID NO: 5) is identical to the nucleotide sequence at positions 8690791-8691758 of NCBI accession number NC_000085.7, and the sequence of the downstream homology arm (3′ homology arm, SEQ ID NO: 6) is identical to the nucleotide sequence at positions 8683723-8685086 of NCBI accession number NC_000085.7. The nucleotide sequence of human CD98HC is shown in SEQ ID NO: 7.

Targeting vector construction was carried out e.g., by enzyme cleavage and ligation, and direct synthesis, etc. The constructed targeting vector was first preliminarily verified by enzyme digestion, and then sent to a sequencing company for sequencing verification. The correct targeting vectors verified by sequencing were used in subsequent experiments.

The target sequence determines the targeting specificity of the sgRNA and the efficiency of inducing Cas9 to cleave the target gene. Therefore, target sequence selection and design are crucial for constructing sgRNA expression vectors. sgRNA sequences that recognize 5′ and 3′ target sites were designed and synthesized. sgRNAs with better activity and higher sequence specificity were selected for subsequent experiments. Exemplary target sequences of sgRNAs on the CD98HC gene are as follows:

sgRNA1 target site (SEQ ID NO: 12):
5′-TTTGTAGGCCGGGATGCGGGAGG-3′
sgRNA2 target site (SEQ ID NO: 13):
5′-ACTAAGCAAAAGTTTAGCGCTGG-3′

Restriction sites were added to the 5′ end of the sgRNA and the complementary strand to obtain the forward oligonucleotide and reverse oligonucleotide sequences. After annealing, the annealed products were ligated to the pT7-sgRNA plasmid (the plasmid was first linearized with BbsI) to obtain expression vectors pT7-EGFR-1 and pT7-EGFR-2. The pT7-sgRNA vector was synthesized, which included a DNA fragment containing the T7 promoter and sgRNA scaffold (SEQ ID NO: 14), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-CD98HC-1 and pT7-CD98HC-2 plasmids (using Ambion™ in vitro transcription kit to carry out the transcription according to the method provided in the product instruction) were injected into the cytoplasm or nucleus of fertilized eggs of C57BL/6 mice with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were transferred to a culture medium to culture for a short time and then transplanted into the oviduct of the recipient mouse to produce the genetically modified mice (F0 generation). The mouse population was further expanded by cross-breeding and self-breeding to establish stable homozygous mouse lines.

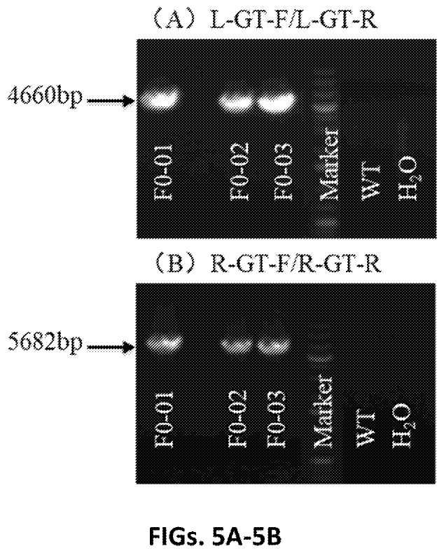

The genotype of the somatic cells of the F0 generation mice can be identified by PCR. The results of some F0 generation mice are shown in FIGS. 5A-5B. As shown by the results of the 5′ primer and 3′ primer, and as further verified by sequencing, the mice numbered F0-01, F0-02, and F0-03 were positive mice. The PCR primers are shown in the table below.

TABLE 3
Primer sequences and recombinant fragment
sizes for F0 genotype PCR detection

| | | | Recombinant |
|---|---|---|---|
| | SEQ ID | Primer sequence | fragment |
| Primer | NO | (5′-3′) | size |
| L-GT- | SEQ ID | AATCTCTTCTGGATG | Mut: |
| F | NO: 15 | CCTGATTCCC | 4660bp |
| L-GT- | SEQ ID | GTTGCAGTGAGCCAA | |
| R-R | NO: 16 | GATCACG | |
| R-GT- | SEQ ID | CATTAGGGACAAAGC | Mut: |
| F | NO: 17 | TTGGGCGAGA | 5682bp |
| R-GT- | SEQ ID | CCTCAGGCTCTGCCT | |
| R | NO: 18 | ATGTAGTTC | |

Among them, the primer L-GT-F is located upstream of the 5′ homology arm, R-GT-R is located downstream of the 3′ homology arm, and both L-GT-R and R-GT-F are located in the human CD98HC sequence.

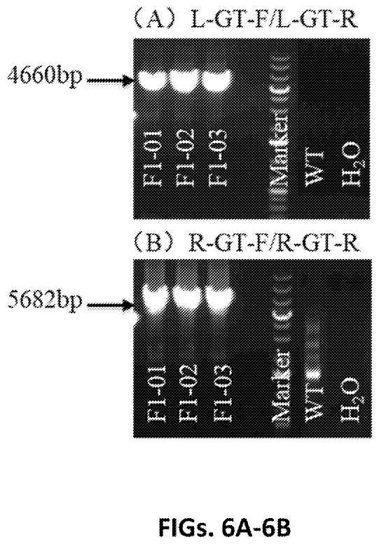

The F0 generation CD98HC humanized mice identified as positive were mated with wild-type mice to obtain F1 generation mice. The same PCR method (primer sequences are listed in the table above) can be used to determine the genotype of the F1 generation mice. The results of some F1 generation mice are shown in FIGS. 6A-6B, which shows three mice (numbered F1-01, F1-02, and F1-03) as positive mice.

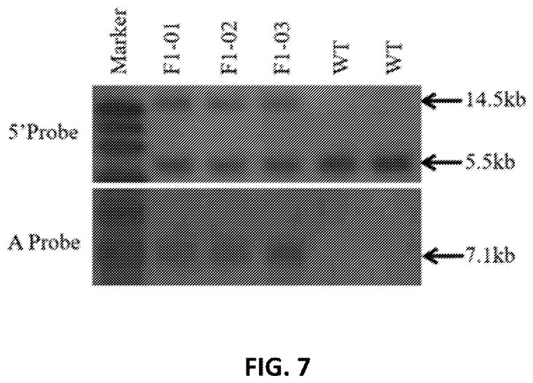

The F1 generation mice identified as positive by PCR were further verified by Southern Blot to confirm whether there was random insertion. Specifically, genomic DNA from the mouse tail was extracted, and digested with restriction enzyme BgLII or SspI. The digested genomic DNA was then transferred to a membrane and hybridized with respective probes. The 5′ probe and the A probe are respectively located in the 5′ homology arm and the human fragment. The probes and the size of target fragment sizes are shown in the table below. The results of some are shown in FIG. 7. The combined results of the A probe and the 5′ probe showed that the three positive mice (numbered F1-01, F1-02, and F1-03) had no random insertion. This shows that the described method can be used to construct CD98HC humanized mice with no random insertion and the genotype in the mice can be stably passaged.

TABLE 4
Lengths of specific probes and target fragments

| | | Wildtype | Recombinant |
|---|---|---|---|
| Endonuclease | Probe | fragment size | fragment size |
| BgLII | 5′ Probe | 14.5 kb | 5.5 kb |
| SspI | A Probe | — | 7.1 kb |

The following primers were used for probe synthesis in Southern Blot assays:

5′Probe-F (SEQ ID NO: 19):
5′-ACATCTGGTTGGTTTGATCCATCTTGT-3′,
5'Probe-R (SEQ ID NO: 20):
5'-GGCCAGAACAGTAGAGAGGAAGGGA-3′;
A Probe-F (SEQ ID NO: 21):
5'-CACTGCACCCGGCCATCAGCAT-3′;
A Probe-R (SEQ ID NO: 22):
5'-CTCACACATACACTGTTGCCCCTCAG-3′;

The F1 generation heterozygous mice identified as positive were crossed with each other, to obtain the F2 generation homozygous CD98HC humanized mice.

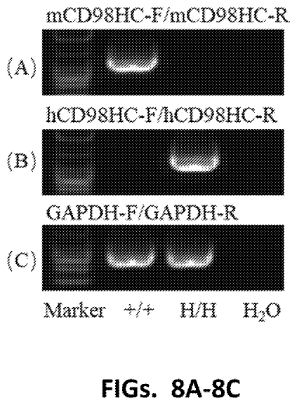

The expression of humanized CD98HC mRNA in positive mice was confirmed by RT-PCR. Specifically, one 7-week-old female wild-type C57BL/6 mouse (+/+) and one CD98HC gene humanized homozygote (H/H) prepared in this practice were selected, and the kidney tissues were collected after euthanasia by neck dislocation. RT-PCR detection was performed using the primer sequences shown in the table below. The results of some are shown in FIGS. 8A-8C. In wild-type C57BL/6 mice, only mouse CD98HC mRNA was detected, and human CD98HC mRNA was not detected. In CD98HC humanized homozygous mice, only human CD98HC mRNA was detected, and murine CD98HC mRNA was not detected.

TABLE 5
RT-PCR primer sequences and target fragment size

| | SEQ | Primer sequence | Fragment |
|---|---|---|---|
| Primer | ID NO | (5′-3′) | size |
| mCD98HC- | SEQ ID | AGGTGAAGGGCCTGGTGTTA | WT: |
| F | NO: 23 | | 724bp |
| mCD98HC- | SEQ ID | ATGCTGGACTCATTCCACGG | |
| R | NO: 24 | | |
| hCD98HC- | SEQ ID | CTGAAGGGGCGTCTCGATTA | Mut: |
| F | NO: 25 | | 784bp |
| hCD98HC- | SEQ ID | CATCCCACAGCATGACTGGA | |
| R | NO: 26 | | |
| GAPDH- | SEQ ID | TCACCATCTTCCAGGAGCGAGA | WT: |
| F | NO: 27 | | 479bp |
| GAPDH- | SEQ ID | GAAGGCCATGCCAGTGAGCTT | |
| R | NO: 28 | | |

The expression of humanized CD98HC protein in positive mice was confirmed by flow cytometry. Specifically, a 7-week-old female wild-type C57BL/6 mouse (WT) and a CD98HC gene humanized homozygous mouse (H/H) prepared in this example were respectively selected, and spleen tissues were obtained after euthanasia by neck dislocation. Single cell suspension was prepared and stained with: Brilliant Violet 510™ anti-mouse CD45 Antibody (mCD45), APC anti-mouse CD98 Antibody (mCD98), FITC anti-human CD98 Antibody (hCD98), PerCP/Cy5.5 anti-mouse TCR β chain Antibody (mTCR β), Brilliant Violet 605™ anti-mouse CD19 Antibody (mCD19), Zombie NIR™ Fixable Viability Kit and Purified anti-mouse CD16/32. The stained cells were analyzed by flow cytometry, to determine the percentages of humanized CD98HC (hCD98HC) positive cells and murine CD98HC (mCD98HC) positive cells in T cells and B cells. As controls, mouse isotype control antibody (APC Rat IgG2a, κ Isotype Ctrl Antibody) and human isotype control antibody (FITC Mouse IgG1, κ Isotype Ctrl (FC) Antibody) were used. mCD45+ mTCR β+ cells were identified as T cells. mCD45+ mCD19+ cells were identified as B cells. The results are shown in the table below.

TABLE 6
Flow cytometry detection results of
CD98HC protein on T cells and B cells

| | T cell | | | B cell | | |
|---|---|---|---|---|---|---|
| | Isotype | | | Isotype | | |
| | control | WT | H/H | control | WT | H/H |
| mCD98HC | 0.44% | 14.7% | 0.27% | 0.73% | 17.2% | 0.30% |
| hCD98HC | 0.66% | 0.45% | 27.6% | 0.97% | 0.82% | 27.1% |

As shown in the table above, in the T cells and B cells of CD98HC gene humanized homozygous mice, the proportions of humanized CD98HC protein-positive cells were 27.6% and 27.1%, respectively, indicating that the CD98HC humanized mice constructed in this example can successfully express humanized CD98HC protein.

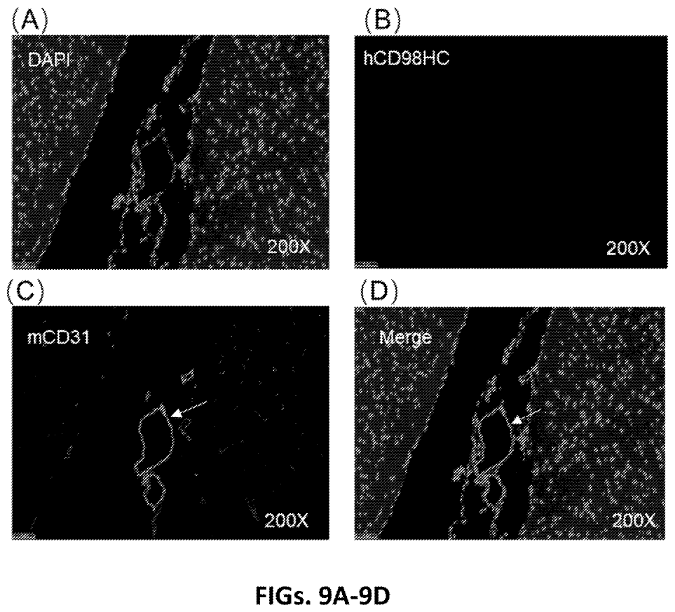

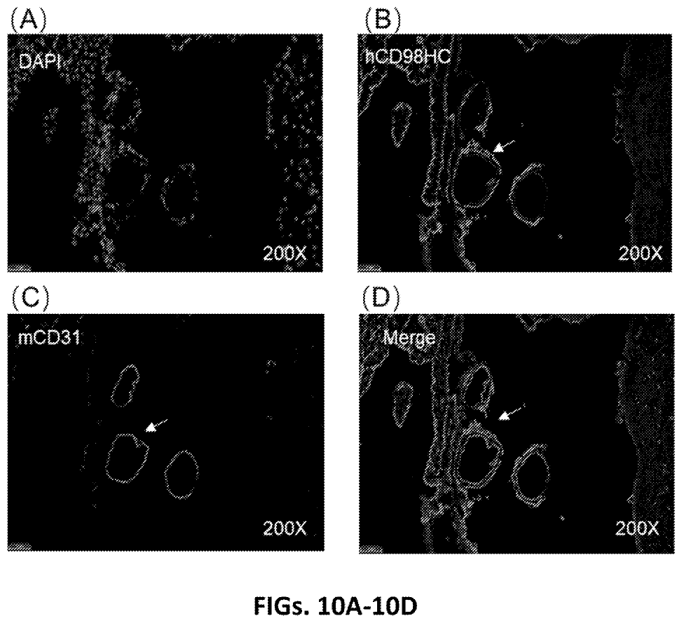

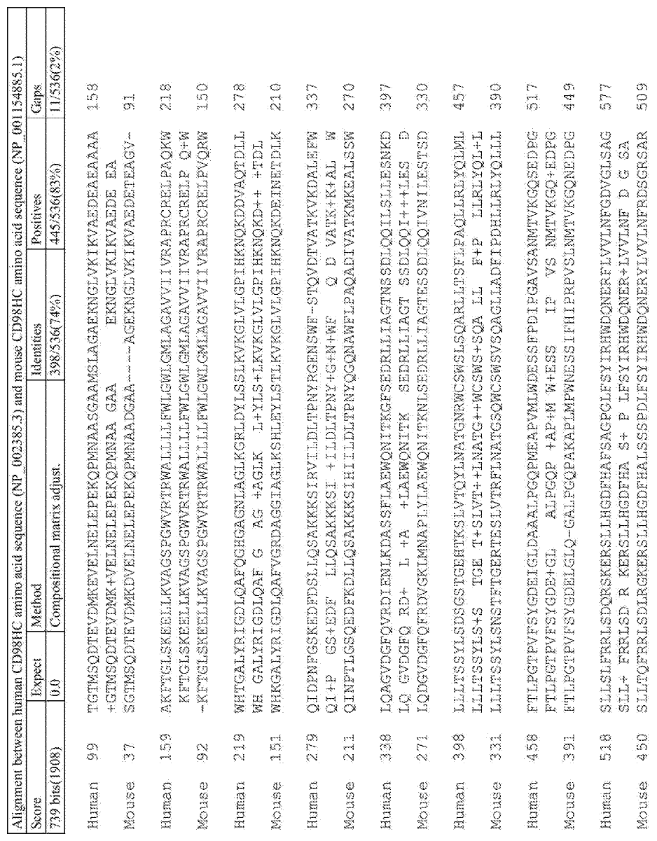

Further, immunofluorescence (IF) staining was used to detect the expression of humanized CD98HC protein in mouse brain. Specifically, three 8-week-old female C57BL/6 wild-type mice (+/+) and three CD98HC humanized homozygous mice (H/H) prepared in this example were selected. After euthanasia by neck dislocation, brain tissues were collected to made paraffin sections, and stained by a anti-mouse CD31 antibody (EPR17259) and an anti-human CD98HC antibody (4F2hc/CD98 (D3F9D) XP® Rabbit mAb). Exemplary IF results are shown in FIGS. 9A-9D and FIGS. 10A-10D. FIGS. 9A-9D show the protein expression in the brain of C57BL/6 wild-type mice. FIGS. 10A-10D show the protein expression in the brain of CD98HC humanized homozygous mice. DAPI is stained for mouse brain cells. Merge indicates the combined result.

In C57BL/6 mice, mCD31+ microvascular endothelial cells (mCD31, indicated by arrows) were detected, but hCD98HC+ microvascular endothelial cells (hCD98HC) were not seen. In CD98HC homozygous mice mCD31+ microvascular endothelial cells and hCD98HC+ microvascular endothelial cells (hCD98HC, indicated by arrows) were both detected. This indicates that the CD98HC humanized mice can successfully express humanized CD98HC protein.

### Example 2: Generation of Double- or Multi-Gene Humanized Mice

The CD98HC humanized mice generated using the methods described herein can also be used to generate double- or multi-gene humanized mouse models. For example, in Example 1, the embryonic stem (ES) cells for blastocyst microinjection can be selected from mice comprising other genetic modifications such as modified (e.g., human or humanized) PD-1, PD-L1, LAG3, 4-1BB, CD40, CTLA4, CD147, IL6R, IL17, CD3, CD28, and/or CD38 genes. Alternatively, embryonic stem cells from CD98HC humanized mice described herein can be isolated, and gene recombination targeting technology can be used to obtain double-gene or multi-gene-modified mouse models. In addition, it is also possible to breed the homozygous or heterozygous CD98HC humanized mice obtained by the methods described herein with other genetically modified homozygous or heterozygous mice, and to screen the offsprings. According to Mendel's law, it is possible to generate double-gene or multi-gene modified heterozygous mice comprising a modified (e.g., human or humanized) CD98HC gene and other genetic modifications. The heterozygous mice can then be bred with each other to obtain homozygous double-gene or multi-gene modified mice. These double-gene or multi-gene modified mice can be used for in vivo testing of drugs targeting human EGFR and other targets.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human animal whose genome comprises at least one chromosome comprising a sequence encoding a human or chimeric cluster of differentiation 98 heavy chain (CD98HC).

2. The animal of claim 1, wherein the sequence encoding the human or chimeric CD98HC is operably linked to an endogenous regulatory element at the endogenous CD98HC gene locus in the at least one chromosome.

3. The animal of claim 1 or 2, wherein the sequence encoding a human or chimeric CD98HC comprises a sequence encoding an amino acid sequence that is at least 70%, 75%, 80%, 85%, 90%, 95%, 99%, or 100% identical to human CD98HC (NP_002385.3 (SEQ ID NO: 2)).

4. The animal of claim 1 or 2, wherein the sequence encoding a human or chimeric CD98HC comprises a sequence encoding an amino acid sequence that is at least 70%, 75%, 80%, 85%, 90%, 95%, 99%, or 100% identical to SEQ ID NO: 11.

5. The animal of claim 1 or 2, wherein the sequence encoding a human or chimeric CD98HC comprises a sequence encoding an amino acid sequence that is at least 70%, 75%, 80%, 85%, 90%, 95%, 99%, or 100% identical to amino acids 215-630 of SEQ ID NO: 2.

6. The animal of any one of claims 1-5, wherein the animal is a mammal, e.g., a monkey, a rodent, a mouse, or a rat.

7. The animal of any one of claims 1-6, wherein the animal is a mouse.

8. The animal of any one of claims 1-7, wherein the animal does not express endogenous CD98HC or expresses a decreased level of endogenous CD98HC as compared to CD98HC expression level in a wild-type animal.

9. The animal of any one of claims 1-8, wherein the animal has one or more cells expressing human or chimeric CD98HC.

10. The animal of any one of claims 1-9, wherein the animal has one or more cells expressing human or chimeric CD98HC, and the expressed human or chimeric CD98HC can interact with a human integrin, activating downstream signaling pathways.

11. The animal of any one of claims 1-9, wherein the animal has one or more cells expressing human or chimeric CD98HC, and the expressed human or chimeric CD98HC can interact with an endogenous integrin, activating downstream signaling pathways.

12. A genetically-modified, non-human animal, wherein the genome of the animal comprises a replacement of a sequence encoding a region of endogenous CD98HC with a sequence encoding a corresponding region of human CD98HC at an endogenous CD98HC gene locus.

13. The animal of claim 12, wherein the sequence encoding the corresponding region of human CD98HC is operably linked to an endogenous regulatory element at the endogenous CD98HC locus, and one or more cells of the animal expresses a human or chimeric CD98HC.

14. The animal of claim 12 or 13, wherein the animal does not express endogenous CD98HC or expresses a decreased level of endogenous CD98HC as compared to CD98HC expression level in a wild-type animal.

15. The animal of any one of claims 12-14, wherein the replaced sequence encodes all or a portion of the extracellular region of CD98HC.

16. The animal of any one of claims 12-15, wherein the animal has one or more cells expressing a chimeric CD98HC having an extracellular region, a transmembrane region, and a cytoplasmic region, wherein the extracellular region comprises a sequence that is at least 50%, 60%, 70%, 80%, 90%, 95%, or 99% identical to the extracellular region of human CD98HC (NP_002385.3 (SEQ ID NO: 2)).

17. The animal of claim 16, wherein the extracellular region of the chimeric CD98HC has a sequence that has at least 10, 20, 30, 40, 50, 60, 70, 80, 90, 100, 110, 120, 130, 140, 150, 160, 170, 180, 190, 200, 250, 300, 350, 400, 410, or 416 contiguous amino acids that are identical to a contiguous sequence present in the extracellular region of human CD98HC (e.g., amino acids 215-630 of SEQ ID NO: 2).

18. The animal of claim 16, wherein the chimeric CD98HC has a sequence that is at least 10, 20, 30, 40, 50, 60, 70, 80, 90, 100, 110, 111, 112, 113, or 114 contiguous amino acids that are identical to a contiguous sequence present in the cytoplasmic region of endogenous CD98HC (e.g., amino acids 1-114 of SEQ ID NO: 1).

19. The animal of any one of claims 12-18, wherein the sequence encoding a region of endogenous CD98HC comprises exon 1, exon 2, exon 3, exon 4, exon 5, exon 6, exon 7, exon 8, exon 9, and/or exon 10, or a part thereof, of the endogenous CD98HC gene.

20. The animal of claim 19, wherein the animal is a mouse.

21. The animal of any one of claims 12-20, wherein the animal is heterozygous with respect to the replacement at the endogenous CD98HC gene locus.

22. The animal of any one of claims 12-20, wherein the animal is homozygous with respect to the replacement at the endogenous CD98HC gene locus.

23. A method for making a genetically-modified, non-human animal, comprising:
replacing in at least one cell of the animal, at an endogenous CD98HC gene locus, a sequence encoding a region of endogenous CD98HC with a sequence encoding a corresponding region of human CD98HC.

24. The method of claim 23, wherein the sequence encoding the corresponding region of human CD98HC comprises exon 1, exon 2, exon 3, exon 4, exon 5, exon 6, exon 7, exon 8, exon 9, exon 10, exon 11, and/or exon 12, or a part thereof, of a human CD98HC gene.

25. The method of claim 23 or 24, wherein the sequence encoding the corresponding region of human CD98HC comprises a portion of exon 4 (e.g., at least 50 bp), exons 5-11, and a portion of exon 12 (e.g., at least 200 bp), of a human CD98HC gene.

26. The method of any one of claims 23-25, wherein the sequence encoding the corresponding region of human CD98HC encodes amino acids 215-630 of SEQ ID NO: 2.

27. The method of any one of claims 23-26, wherein the region comprises all or a portion of the extracellular region of CD98HC.

28. The method of any one of claims 23-27, wherein the sequence encoding a region of endogenous CD98HC comprises exon 1, exon 2, exon 3, exon 4, exon 5, exon 6, exon 7, exon 8, exon 9, and/or exon 10, or a part thereof, of the endogenous CD98HC gene.

29. The method of any one of claims 23-28, wherein the animal is a mouse, and the sequence encoding a region of endogenous CD98HC comprises a portion of exon 2 (e.g., at least 50 bp), exons 3-9, and a portion of exon 10 (e.g., at least 200 bp) of the endogenous CD98HC gene.

30. A non-human animal comprising at least one cell comprising a nucleotide sequence encoding a humanized CD98HC polypeptide, wherein the humanized CD98HC polypeptide comprises at least 200 contiguous amino acid residues that are identical to the corresponding contiguous amino acid sequence of a human CD98HC extracellular region, wherein the animal expresses the humanized CD98HC polypeptide.

31. The animal of claim 30, wherein the humanized CD98HC polypeptide has at least 200, 250, 300, 350, 400, 410, or 416 contiguous amino acid residues that are identical to the corresponding contiguous amino acid sequence of human CD98HC extracellular region (e.g., amino acids 215-630 of SEQ ID NO: 2).

32. The animal of claim 30 or 31, wherein the humanized CD98HC polypeptide has at least 10, 20, 30, 40, 50, 60, 70, 80, 90, 100, 110, 120, 130, 140, or 146 contiguous amino acid residues that are identical to the contiguous amino acid sequence of endogenous CD98HC (e.g., amino acids 1-146 of SEQ ID NO: 1).

33. The animal of any one of claims 30-32, wherein the humanized CD98HC polypeptide comprises a sequence that is at least 90%, 95%, or 99% identical to amino acids 215-630 of SEQ ID NO: 2.

34. The animal of any one of claims 30-33, wherein the nucleotide sequence is operably linked to an endogenous CD98HC regulatory element of the animal.

35. The animal of any one of claims 30-34, wherein the chimeric CD98HC polypeptide comprises an endogenous CD98HC transmembrane region and/or an endogenous CD98HC cytoplasmic region.

36. The animal of any one of claims 30-35, wherein the nucleotide sequence is integrated to an endogenous CD98HC gene locus of the animal.

37. The animal of any one of claims 30-36, wherein the humanized CD98HC polypeptide has at least one mouse CD98HC activity and/or at least one human CD98HC activity.

38. A method of making a genetically-modified animal cell that expresses a chimeric CD98HC, the method comprising:
replacing at an endogenous CD98HC gene locus, a nucleotide sequence encoding a region of endogenous CD98HC with a nucleotide sequence encoding a corresponding region of human CD98HC, thereby generating a genetically-modified animal cell that includes a nucleotide sequence that encodes the chimeric CD98HC, wherein the animal cell expresses the chimeric CD98HC.

39. The method of claim 38, wherein the animal is a mouse.

40. The method of claim 38 or 39, wherein the chimeric CD98HC comprises a human or humanized CD98HC extracellular region; and a transmembrane and/or a cytoplasmic region of mouse CD98HC.

41. The method of claim 40, wherein the chimeric CD98HC comprises an amino acid sequence that is at least 50%, 60%, 70%, 80%, 90%, 95%, or 99% identical to the amino acid sequence set forth in SEQ ID NO: 11.

42. The method of any one of 38-41, wherein the nucleotide sequence encoding the chimeric CD98HC is operably linked to an endogenous CD98HC regulatory region, e.g., promoter.

43. The animal of any one of claims 1-22 and 30-37, wherein the animal further comprises a sequence encoding an additional human or chimeric protein.

44. The animal of claim 43, wherein the additional human or chimeric protein is selected from the group consisting of: programmed cell death protein 1 (PD-1), programmed cell death ligand 1 (PD-L1), lymphocyte-activation gene 3 (LAG3), tumor necrosis factor receptor superfamily member 9 (4-1BB), CD40, cytotoxic T-lymphocyte-associated protein 4 (CTLA4), CD147, Interleukin 6 receptor (IL6R), Interleukin 17 (IL17), CD3, CD28, and CD38.

45. The method of any one of claims 23-29 and 38-42, wherein the animal or mouse further comprises a sequence encoding an additional human or chimeric protein.

46. The method of claim 45, wherein the additional human or chimeric protein is selected from the group consisting of: PD-1, PD-L1, LAG3, 4-1BB, CD40, CTLA4, CD147, IL6R, IL17, CD3, CD28, and CD38.

47. A method of determining effectiveness of a therapeutic agent for the treatment of cancer, comprising:
a) administering the therapeutic agent to the animal of any one of claims 1-22, 30-37, 43, and 44, wherein the animal has a tumor; and
b) determining inhibitory effects of the therapeutic agent to the tumor.

48. The method of claim 47, wherein the therapeutic agent is an anti-CD98HC antibody.

49. The method of claim 47 or 48, wherein the tumor comprises one or more cancer cells that are injected into the animal.

50. The method of any one of claims 47-49, wherein determining inhibitory effects of the anti-CD98HC antibody to the tumor involves measuring the tumor volume in the animal.

51. The method of any one of claims 47-50, wherein the cancer is lymphoma, bladder cancer, breast cancer, lung cancer, squamous cell carcinoma, intestinal adenoma, or leukemia.

52. A method of determining effectiveness of an anti-CD98HC antibody and an additional therapeutic agent for the treatment of cancer, comprising
a) administering the anti-CD98HC antibody and the additional therapeutic agent to the animal of any one of claims 1-22, 30-37, 43, and 44, wherein the animal has a tumor; and
b) determining inhibitory effects on the tumor.

53. The method of claim 52, wherein the animal further comprises a sequence encoding a human or chimeric PD-1, a human or chimeric PD-L1, and/or a human or chimeric CTLA4.

54. The method of claim 52 or 53, wherein the additional therapeutic agent is an anti-PD-1 antibody, an anti-PD-L1 antibody, or an anti-CTLA4 antibody.

55. The method of any one of claims 52-54, wherein the tumor comprises one or more tumor cells that express PD-L1.

56. The method of any one of claims 52-55, wherein the tumor comprises one or more cancer cells that are injected into the animal.

57. The method of any one of claims 52-56, wherein determining inhibitory effects of the treatment involves measuring the tumor volume in the animal.

58. The method of any one of claims 56-57, wherein the animal has lymphoma, bladder cancer, breast cancer, lung cancer, squamous cell carcinoma, intestinal adenoma, or leukemia.

59. A method of determining effectiveness of a therapeutic agent for treatment an immune disorder, comprising:
a) administering the therapeutic agent to the animal of any one of claims 1-22, 30-37, 43, and 44, wherein the animal has the immune disorder; and
b) determining effects of the therapeutic agent to the immune disorder.

60. A method of determining effectiveness of a therapeutic agent for reducing an inflammation, comprising:
a) administering the therapeutic agent to the animal of any one of claims 1-22, 30-37, 43, and 44, wherein the animal has the inflammation; and
b) determining effects of the therapeutic agent to the inflammation.

61. A method of determining effectiveness of a therapeutic agent for treating an autoimmune disease, comprising:
a) administering the therapeutic agent to the animal of any one of claims 1-22, 30-37, 43, and 44, wherein the animal has the autoimmune disease; and
b) determining effects of the therapeutic agent to the autoimmune disease.

62. The method of claim 61, wherein the autoimmune disease is type 1 diabetes or multiple sclerosis.

63. A method of determining toxicity of a therapeutic agent comprising:
a) administering the therapeutic agent to the animal of any one of claims 1-22, 30-37, 43, and 44; and
b) determining effects of the therapeutic agent to the animal.

64. The method of claim 63, wherein the therapeutic agent is an anti-CD98HC antibody.

65. The method of claim 63 or 64, wherein determining effects of the therapeutic agent to the animal involves measuring the body weight, red blood cell count, hematocrit, and/or hemoglobin of the animal.

66. A protein comprising an amino acid sequence, wherein the amino acid sequence is one of the following:
(a) an amino acid sequence set forth in SEQ ID NO: 1, 2, or 11;
(b) an amino acid sequence that is at least 90% identical to SEQ ID NO: 1, 2, or 11;
(c) an amino acid sequence that is at least 91%, 92%, 93%, 94%, 95%, 96%, 97%, 98%, or 99% identical to SEQ ID NO: 1, 2, or 11;
(d) an amino acid sequence that is different from the amino acid sequence set forth in SEQ ID NO: 1, 2, or 11 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, or 11.

67. A nucleic acid comprising a nucleotide sequence, wherein the nucleotide sequence is one of the following:
(a) a sequence that encodes the protein of claim 67;
(b) SEQ ID NO: 3, 4, 5, 6, 7, 8, 9, 10, 12, or 13;
(c) a sequence that is at least 90% identical to SEQ ID NO: 3, 4, 5, 6, 7, 8, 9, 10, 12, or 13; and
(d) a sequence that is at least 91%, 92%, 93%, 94%, 95%, 96%, 97%, 98%, or 99% identical to SEQ ID NO: 3, 4, 5, 6, 7, 8, 9, 10, 12, or 13.

68. A cell comprising the protein of claim 66 and/or the nucleic acid of claim 67.

69. An animal comprising the protein of claim 66 and/or the nucleic acid of claim 67.
